(19) United States
(12) Patent Application Publication
(10) Pub. No.: US 2022/0016164 A1
(43) Pub. Date: Jan. 20, 2022

(54) PHARMACEUTICAL COMPOSITION FOR USE IN THE TREATMENT OF PANCREATIC CANCER

(71) Applicant: Amphera B.V., Hertogenbosch (NL)

(72) Inventors: Joachim AERTS, Rotterdam (NL); Floris DAMMEIJER, Rotterdam (NL)

(21) Appl. No.: 17/443,623

(22) Filed: Jul. 27, 2021

Related U.S. Application Data
(63) Continuation of application No. PCT/NL2020/050042, filed on Jan. 28, 2020.

(30) Foreign Application Priority Data
Jan. 28, 2019 (NL) 2022464
Dec. 19, 2019 (NL) 202416

(51) Int. Cl. A61K 35/15; C07K 16/28; C12N 1/06; A61K 45/06; A61K 39/00

(52) U.S. Cl. CPC A61K 35/15; C07K 16/2878; C12N 1/06; A61K 2039/5154; A61K 39/001106; A61K 2039/852; A61K 45/06

(57) ABSTRACT

The present invention relates to a method for the treatment of pancreatic cancer comprising administering to a patient in need thereof a CD40 agonist in combination with dendritic cells loaded with a lysate of mesothelioma cells. A further aspect of the present invention relates to dendritic cells loaded with a lysate of mesothelioma cells for use in the treatment of pancreatic cancer. A last aspect of the present invention relates to a pharmaceutical composition comprising such loaded dendritic cells.

### CROSS-REFERENCE TO RELATED APPLICATIONS

The present application is a Continuation of International Patent Application No. PCT/NL2020/050042, filed Jan. 28, 2020, which claims priority to (i) Netherlands Patent Application No. 2022464 filed Jan. 28, 2019; and (ii) Netherlands Patent Application No. 2024516 filed Dec. 19, 2019; the entire contents of all of which are hereby incorporated by reference.

### FIELD OF THE INVENTION

The present invention relates to a method for the treatment of pancreatic cancer with dendritic cells loaded with a lysate of mesothelioma tumour cells in combination with a CD40 agonist. The present invention further relates to loaded dendritic cells and a pharmaceutical composition thereof for use in the treatment of pancreatic cancer in combination with a CD40 agonist.

### BACKGROUND OF THE INVENTION

The annual incidence of patients developing pancreatic cancer in the Netherlands is approximately 3500 (1). In 2020, pancreatic cancer is expected to be the second leading cause of cancer-related death (2). The 1-year overall survival (OS) for pancreatic cancer in the Netherlands is 20%; 5-year OS is only 3% (3). The vast majority of patients presents with either locally advanced or metastatic disease, which excludes them from curative surgery. Only 15-25% of all pancreatic cancer patients are eligible to undergo surgical resection (4). However, ten years after resection, OS is still only 4%, demonstrating that cure is rare (5). Apparently, the vast majority of patients with (borderline) resectable pancreatic cancer according to imaging techniques have occult metastatic disease. Adjuvant chemotherapy after surgical resection does improve the median overall survival to 28 months in contrast to chemo-radiotherapy which was found to marginally improve pancreatic cancer survival (6, 7). However, even with the new regimens of chemotherapy, long-term survival is still exceptional. Since recurrence rates are extremely high after resection, we are in need of new treatments in order to curb the progression of pancreatic cancer.

The potential to harness the potency and the specificity of the immune system underlies the growing interest in cancer immunotherapy. One approach to activate the patient's immune system uses dendritic cell based immunotherapy. Dendritic cell based immunotherapy aims to boost the immune system of cancer patients by enhancing tumour antigen recognition by activating cytotoxic T-cells and thus generating anti-tumour specific responses.

In this regard it is well known that dendritic cells are highly mobile and extremely potent antigen presenting cells located at strategic places where the body comes in contact with its environment. In these locations they pick up antigens and transport them to the secondary lymphoid organs where they instruct and control activation of natural killer cells, B and T-lymphocytes, and efficiently activate them against the antigens. This property makes them attractive candidates for use in therapeutic strategies against cancer. Furthermore, dendritic cells can be generated in large numbers ex vivo.

Cancer induces a highly immunosuppressive tumour microenvironment (TME) leading to the dysfunction of multiple immune effector cells (8, 9). For instance, cytokines related to anti-inflammatory Th2 phenotype and immune-suppressive regulatory T cells are elevated in peripheral blood in patients with pancreatic cancer compared to healthy controls (10, 11), whereas the accumulation of cytotoxic CD8 T cells is lagging behind (12). This causes a non-cytotoxic T-cell infiltrated tumour, and may explain the low response rate of immune checkpoint antibodies like PD-1/PD-L1 (13). In pancreatic cancer, early trials indeed show disappointing results with these antibodies, pointing to the need for a more basal activation of the immune system (14-16). The induction of robust immune effector cells could enhance CD8 T cell infiltration and shift the balance in favour of an anti-cancer response. One approach to activate the patient's immune system and induce tumour directed cytotoxic T-cells is by using cancer vaccines. Cancer vaccines have yielded promising results in several preclinical and clinical studies (17). In complex immunological tumours, cellular therapies seem more effective than other types of vaccination (18). Various types of cellular vaccinations have been tested in pancreatic cancer in the setting of phase I or II trials.

Below, we will discuss the most promising therapy types in pancreatic cancer (i.e. tumour cell-based vaccination, adoptive T-cell transfer and dendritic cell vaccination).

### Tumour Cell-Based Vaccines

In pancreatic cancer only two types of tumour cell-based vaccines (without adoptive cell transfer) are currently known. Their goal is to prime a robust immune response by activating different immune effector cells. Algenpantucel-L consists of two irradiated human pancreatic cancer cell lines (HAPa-1 and HAPa-2) which express the murine enzyme α-1,3-galactosyl transferase (α-GT)(19). While two phase III clinical trials with Algenpantucel-L are still ongoing, a recent press release announced failed improvement of OS of Algenpantucel-L versus standard of care in one of these phase III clinical trials. Median OS in the intervention group was 27.3 months while the control group with standard of care showed a median OS of 30.4 months (20).

The second tumour cell-based vaccine tested in pancreatic cancer patients is GVAX. The GVAX vaccine is based on irradiated tumour cells modified to express granulocyte-macrophage colony-stimulating factor (GM-CSF) (21, 22). This is combined with CRS-207,

Listeria monocytogenes engineered to express mesothelin. Some patients treated with GVAX/CRS-207 and radiochemotherapy developed an immune response against mesothelin and showed an increase in progression free survival and OS (21, 23). However, the phase 2b trial ECLIPSE did not meet the primary endpoint of an improvement of OS for patients with pancreatic cancer (24).

### Adoptive T Cell Transfer

Tumour-specific effector CD8+T cells are considered to be the final, and vital, step in immune-mediated cancer eradication. Therefore, adoptive cell transfer (ACT) with effector T cells has been developed which includes tumour-infiltrating lymphocytes (TIL) therapy and receptor-engineered T cell therapy (25). However, widespread clinical use of TI Ls in solid tumours is limited due to practical barriers. Especially in pancreatic cancer harvesting of tumour cells is extremely challenging due to the prominent desmoplastic stroma present in pancreatic cancer (26, 27). To date, no clinical trial with TIL therapy has been performed in pancreatic cancer patients. Furthermore, lymphocytes can be engineered by introducing genes encoding for anti-tumour alpha-beta T cell receptors (TCRs) or chimeric antigen receptors (CARs) into mature T cells. (28). However, there are some concerns and weaknesses concerning TCR and CAR T-cell therapy. ACT with effector T cells bears the risk of toxicity when targeting antigens are shared by tumours and normal tissue, or when target antigens are highly similar to self-antigens. (29-31) Unexpected lethal toxicities have been observed in a number of trials due to previously unknown cross-reactivity (32-34). Furthermore, results in solid tumours are less encouraging due to the presence of an immune-suppressive micro-environment that may adversely affect recruitment and activation of adoptive CD8 T cells (35).

### Dendritic Cell Vaccination

Dendritic cells (DCs) are the most potent activators of the immune system and play a fundamental role in the effectiveness of cancer vaccines (36). DCs can capture, process and present tumour associated antigens (TAAs) in context of a Major Histocompatibility Complex (MHC) Class I or II (37). Subsequently, DCs can prime naive T cells, memory T cells and B cells which are needed for the induction of a robust anti-cancer response (38, 39). DCs pulsed with TAAs have shown beneficial effect in tumour animal models (40, 41) where they were shown to be essential in eliciting a vigorous anti-cancer response. Clinical studies have shown the safety and efficacy of DC immunotherapy (42, 43). Safety of DC-based immunotherapy in patients with pancreatic cancer was studied in several phase I and II studies. Until now, about 20 clinical DC immunotherapy trials in pancreatic cancer have been performed worldwide. DCs were pulsed with TAAs such as Wilms' tumour 1 (WT-1), MUC-1, carcino-embryonic antigen (CEA), survivin, human telomerase reverse transcriptase (hTERT) or autologous tumour material (44-51) with varying results.

### SUMMARY OF THE INVENTION

A need remains for an efficient curative, palliative, or preventive treatment of pancreatic cancer. This is particularly the case for patients that have not received or are ineligible to surgery or for patients with recurrent pancreas tumours. The current invention provides such treatment for pancreatic cancer by means of a combination therapy of a CD40 agonist and dendritic cells loaded with an allogeneic tumour cell line lysate, or a pharmaceutical composition comprising such dendritic cells loaded with such an allogeneic tumour lysate.

A first aspect of the present invention relates to a method for the treatment of pancreatic cancer comprising administering to a patient in need thereof a CD 40 agonist in combination with dendritic cells loaded with a lysate, wherein the lysate is obtainable by a method comprising the steps of:

i) providing human mesothelioma tumour cells from at least two different mesothelioma tumour cell lines;
ii) inducing necrosis in said tumour cells; and
iii) lysing the necrotic tumour cells, such that a lysate is obtained.

It has surprisingly been found that a lysate of mesothelioma cells, previously successfully used in clinical trials for the treatment of mesothelioma (54), is very useful in the treatment of pancreatic cancers as well, particularly when combined with a CD40 agonist.

A second aspect of the present invention relates to dendritic cells loaded with a lysate for use in the treatment of pancreatic cancer, wherein said dendritic cells are administered to a patient in need thereof in combination with a CD40 agonist and wherein the lysate is obtainable by a method comprising the steps of:

i) providing human mesothelioma tumour cells from at least two different mesothelioma tumour cell lines;
ii) inducing necrosis in said tumour cells; and
iii) lysing the necrotic tumour cells, such that a lysate is obtained.

A third aspect of the present invention relates to a pharmaceutical composition for use in the treatment of pancreatic cancer together with a CD40 agonist, wherein said composition is obtainable by a method comprising the steps of:

i) providing allogeneic mesothelioma tumour cells from at least two different cell lines, and preparing a lysate thereof;
ii) providing dendritic cells;
iii) loading the dendritic cells with the lysate of tumour cells and, optionally, providing and adding a pharmaceutically acceptable carrier.

### DEFINITIONS

The term “antigen” as used herein has its conventional meaning and refers to a molecule capable of inducing an immune response. Within the context of the present invention the antigen may be a protein or a fragment thereof, such as a (poly)peptide representing an epitope of said protein. It is however also possible that the antigen used is an artificial peptide or a peptidomimetic, e.g., by incorporating rigid unnatural amino acids, such as 3-aminobenzoic acid, into peptides to make the peptide backbone rigid. The antigens used in the present invention are preferably proteins or parts thereof obtained or derived from a tumour-cell.

The term “epitope” as used herein has its conventional meaning and refers to the part of an antigen that is recognized by the immune system, in particular by antibodies, B cells, or T cells. Within the context of the present invention the antigen is a protein and the epitope is part thereof (i.e. a (poly)peptide, fragment or aggregate thereof).

The term “cancer” as used herein has its conventional meaning and refers to the broad class of disorders characterized by hyper-proliferative cell growth in vivo.

The term “mesothelioma cancer cells” or “mesothelioma tumour cells” as used herein has its conventional meaning and refers to cells from malignant mesothelioma.

The term “pancreatic cancer cells” or “pancreatic tumour cells” as used herein has its conventional meaning and refers to cells from a malignant pancreatic cancer.

The term “for use in the treatment of pancreatic cancer” as used herein has its conventional meaning and refers to the reduction of the size of a pancreatic tumour or number of pancreatic cancer cells, cause a pancreatic cancer to go into remission or prevent or delay further growth in size or cell number of pancreatic cancer cells.

The term “cold tumour” as used herein has its conventional meaning and refers to a tumour wherein there is no or minimal presence of infiltrating cytotoxic T-cells.

The term “hot tumour” as used herein has its conventional meaning and refers to a tumour wherein there is a considerable presence of cytotoxic T-cells either active or inactivated via for example the different immune checkpoints.

The term “progression free survival” (PFS) as used herein has its conventional meaning and refers to the time from treatment (or randomization) to first disease progression or death.

The term “overall survival” (OS) as used herein has its conventional meaning and refers to the patient remaining alive from randomization or from initial diagnosis.

### BRIEF DESCRIPTION OF THE FIGURES

FIG. 1: Experimental setup Example 3. Immunocompetent C57bl/6 mice were treated with DC-vaccines consisting of monocyte-derived DCs loaded with either pancreatic cancer lysate (KPC-3) or with mesothelioma lysate (AE17). An untreated group was also included. Subsequently, a pancreatic tumour was induced with the KPC-3 tumour cell line and tumour growth was followed.

FIG. 2: Tumour growth following DC vaccination. (A) Tumour size measured over time of untreated and treated mice. (B) Tumour growth curve per mouse. N=8 per group. Significance was determined using the non-parametric Mann-Whitney U test. Data presented as the mean±s.e.m. *P<0.015. KPC-3=pancreatic cancer lysate-DC therapy, AE17=mesothelioma lysate-DC therapy.

FIG. 3: End-stage analysis following DC vaccination. (A) CD3⁺, CD4⁺ and CD8⁺ TILs as a percentage of CD45⁺ alive subset of treated and untreated mice 27 days following DC vaccinations, determined by flow cytometry. (B) Percentages of CD44 or Ki67-positive CD4⁺ and CD8⁺ TILs of treated and untreated mice. (C) CD3⁺, CD4⁺ and CD8⁺ T-cells as a percentage of CD45⁺ alive subset in peripheral blood of treated and untreated mice. (D) CD44⁺CD62L⁻ subset or Ki67 positivity of CD4⁺ and CD8⁺ peripheral blood T-cells of treated and untreated mice. (E) Percentage of PD-1⁺ TIM-3⁻LAG⁻ within CD8⁺ TILs. (F) Tregs (CD4⁺CD25⁺FoxP3⁺) as a percentage of CD45 alive subset in tumours. All non-Treg CD4⁺ subsets are FoxP3⁻. N=8 per group. Significance was determined using the non-parametric Mann-Whitney U test. Data presented as the mean±s.e.m. *P<0.05, **P<0.01, ***P<0.001.

FIG. 4: Tumour-reactive T-cell responses following DC treatment. CD8⁺ MACS-purified fresh splenocytes (assay performed at the day of sacrifice, day 27) were co-cultured with KPC-3 tumour cells. KPC-3 tumour cells were first stimulated overnight with INFγ (40 U/ml), after which 100.000 cells were seeded together with CD8⁺ T-cells at a ratio of 1:1 in a 96 wells flat bottom plate and incubated at 37° C. in a humidified atmosphere at 5% CO₂ for 5 hours together with 10 μg/ml CD107a-FITC (BD Bioscience). After one hour, the protein transport inhibitor Golgi stop™ was added (BD Bioscience). For the markers granzyme B and TNFα splenocytes were stimulated with 50 ng/ml phorbol 12 myristate 13-acetate (PMA) and 500 ng/ml ionomycin (Sigma) for 5 hours. N=8 per group. Significance was determined using the non-parametric Mann-Whitney U test. Data presented as the mean±s.e.m. **P<0.01, ***P<0.001.

FIG. 5: Experimental setup Example 4. KPC-3 C57Bl/6 mice were treated with either unloaded (i.e. in the absence of tumour lysate) but matured DCs (stimulated with CpG) or DCs that were matured and loaded with the mesothelioma AE17 lysate.

FIG. 6: Tumour growth following DC vaccination. Tumour volume measured over time of mice treated with DCs pulsed with and without mesothelioma lysate. N=7 per group. Significance was determined using the non-parametric Mann-Whitney U test. Data presented as the mean±s.e.m. *P<0.05 **P<0.01.

FIG. 7: Schematic overview Example 5. Tumour and spleen from treated and untreated tumour-bearing mice from Example 4 were snap frozen and stored in single cell suspension respectively. Bone marrow was harvested from wild type non-tumour bearing mice for the culture of mature DCs.

FIG. 8: Tumour-reactive T-cell responses following DC vaccination. Thawed splenocytes from pancreatic tumour-bearing mice were cocultured with GM-CSF cultured DCs that were loaded with 70 ug autologous pancreatic tumour lysate or control lung lysate (depicted on x-axis). 100,000 DCs were co-cultured with splenocytes of either untreated tumour bearing mice (first and fourth bar in each graph), tumour bearing mice treated with unloaded DCs (second and fifth bar in each graph), and tumour bearing mice treated with AE17 loaded DCs (third and sixth bar in each graph) at a ratio of 1:10 in a 96 wells round bottom plate and incubated at 37° C. in a humidified atmosphere at 5% CO₂ for 24 hours. After 20 hours the protein transport inhibitor Golgi Stop™ was added (BD Bioscience) and after 23 hours 10 μg/ml CD107a-FITC (BD Bioscience) was added per well. CD107, Granzyme B, IFNγ and TNFα were determined by flow cytometry. N=5-8 per group. Significance was determined using the non-parametric Mann-Whitney U test. Data presented as the mean±s.e.m. **P<0.01, ***P<0.001.

FIG. 9: Experimental setup Example 8. Immunocompetent C57bl/6 mice were subcutaneously injected with 1*10⁵ pancreatic tumour cells and treated with either DC vaccine, CD40 agonistic monoclonal antibody, or both as indicated in the Figure. On day 5, mice received 1*10⁶ DCs and on days 6 and 12 CD40 agonistic monoclonal antibody or its isotype as indicated in the Figure.

FIG. 10. Tumour growth. (A) Tumour size measured over time of untreated and treated mice with mesothelioma lysate-DC therapy, FGK45 (a CD40 agonistic monoclonal antibody) or both. (B) Tumour volume on day 18 post-tumour injection. N=8 per group. Significance was determined using the non-parametric Mann-Whitney U test. Data presented as the mean±s.e.m. *P<0.05, **P<0.01.

FIG. 11. Peripheral blood analysis following DC vaccination and FGK antibody injection. (A) CD69+ and Ki67+ cells as a percentage of CD4⁺ and CD8⁺ T cells in peripheral blood of treated and untreated mice. (B) CD44⁺CD62L⁻ and CD44⁻CD62L⁺ subsets as a percentage of CD4⁺ and CD8⁺ peripheral blood T-cells of treated and untreated mice. N=8 per group. Significance was determined using the non-parametric Mann-Whitney U test. Data presented as the mean±s.e.m. *P<0.05, **P<0.01, ***P<0.001.

FIG. 12. Endstage tumour analysis. CD3⁺, CD4⁺, CD8⁺ and CD4⁺CD25⁺FoxP3+ TILs as a percentage of CD45⁺ alive subset and absolute cell count per mg tumour of treated and untreated mice at end-stage disease, determined by flow cytometry. N=8 per group. Significance was determined using the non-parametric Mann-Whitney U test. Data presented as the mean±s.e.m. *P<0.05, **P<0.01, ***P<0.001.

FIG. 13. Study protocol. In this figure the study protocol is provided. Mice were administered tumour cells on day 0. Thereafter they received DC vaccinations (AE17) followed by administration of agonistic CD40 antibody FGK45.

FIG. 14 Tumour growth. In this figure the growth of the tumour in said mice is shown. It is clear that the combination therapy resulted in a remarkable decrease of growth of the pancreatic tumours.

FIG. 15 Survival. In this figure a Kaplan Meier curve is shown of the treated mice. From this it is clear that the combination therapy of the invention considerably increased the overall survival period of the mice.

### DETAILED DESCRIPTION OF THE INVENTION

A first aspect of the present invention relates to a method for the treatment of pancreatic cancer comprising administering to a patient in need thereof a CD40 agonist in combination with dendritic cells loaded with a lysate, wherein the lysate is obtainable by a method comprising the steps of:

i) providing human mesothelioma tumour cells from at least two different mesothelioma tumour cell lines;
ii) inducing necrosis in said tumour cells; and
iii) lysing the necrotic tumour cells, such that a lysate is obtained.

A second aspect of the present invention relates to dendritic cells loaded with a lysate for use in the treatment of pancreatic cancer, wherein said dendritic cells are administered to a patient in need thereof in combination with a CD40 agonist and wherein the lysate is obtainable by a method comprising the steps of:

i) providing human mesothelioma tumour cells from at least two different mesothelioma tumour cell lines;

ii) inducing necrosis in said tumour cells; and

iii) lysing the necrotic tumour cells, such that a lysate is obtained.

A third aspect of the present invention relates to a pharmaceutical composition for use in the treatment of pancreatic cancer together with a CD40 agonist, wherein said composition is obtainable by a method comprising the steps of:

i) providing allogeneic mesothelioma tumour cells from at least two different cell lines, and preparing a lysate thereof;
ii) providing dendritic cells;
iii) loading the dendritic cells with the lysate of tumour cells and, optionally, providing and adding a pharmaceutically acceptable carrier.

It has surprisingly been found that with a combination of a CD40 agonist and dendritic cells loaded with a lysate obtainable by the method referred to above (or a pharmaceutical composition thereof) it has become possible to effectively treat pancreatic cancer.

It has particularly become possible to effectively treat patients with unresected pancreas tumours.

It is especially possible with the present invention to treat patients suffering from primary pancreatic cancer, locally advanced pancreatic cancer, metastatic pancreatic cancer or borderline resectable pancreatic cancer.

It has been found that it is particularly advantageous to treat patients suffering from or at risk of metastatic pancreatic cancer. The present invention and the various options described herein may thus be used in patients suffering from metastatic pancreatic cancer.

With “unresected” in this context is meant that the tumour has not been either partly or completely removed by surgery. Such tumour can either be the primary or a metastatic secondary pancreatic tumour.

### The CD40 Agonist

With “CD40 agonist” is meant: an agonist of the cell surface receptor CD40, with potential immunostimulatory and antineoplastic activities. Similar to the endogenous CD40 ligand (CD40L or CD154), a CD40 agonist is preferably able to bind to CD40 on a variety of immune cell types. Binding of the agonist to the CD40 molecule may trigger the cellular proliferation and activation of antigen-presenting cells (APCs), and activation of B cells and T cells, resulting in an enhanced immune response. Particularly preferred are agonistic CD40 monoclonal antibodies (herein also referred to as CD40 agonistic monoclonal antibody), fragments or derivatives thereof, such a single domain antibody (also referred to as nanobody), a single chain antibody, a single chain variable fragment (scFv), a Fab fragment or a F(ab′)2 fragment.

The CD40 agonist to be administered in combination with lysate loaded dendritic cells or composition according to the invention may be a natural CD40 ligand, such as CD40L, or a functional fragment thereof having agonistic properties. The CD40 agonist may also be a monoclonal antibody having agonistic properties, such as, e.g., CP-870, CP-893 (61), CDX-1140, APX005M, RG7876/selicrelumab, ADC-1013/JNJ-64457107, ABBV-428, SEA-CD40 or MED15083 (62) or a functional fragment thereof having agonistic properties. The CD40 agonist may also be a small molecule which has, for instance, been designed to mimic (the effects of) a natural ligand or an agonistic antibodies, such as, e.g., MiniCD40Ls-1 or MiniCD40Ls-2 (63).

Without wishing to be bound by any theory the present inventors believe that in contrast to, for instance, melanoma and non-small cell lung cancer, pancreatic cancers are in general immunological cold tumours. It is thought that the characteristic desmoplastic stroma of established pancreatic adenocarcinomas is contributing to this phenotype acting as a physical as well as an immunosuppressive barrier leading to the exclusion of T cells (64). The current inventors have explored the hypothesis that a CD40 agonist may convert pancreatic adenocarcinomas into immunological hot tumours by T-cell-dependent and T-cell-independent mechanisms.

It has thereby also been observed that the combination therapy according to the present invention is able to also upregulate expression of VEGFa, adm and Flt1 compared to mice treated with a CD40 agonist only. This is an indication that angiogenesis and vascular formation is triggered, which promotes immune cell infiltration into the tumours.

The present inventors observed a surprisingly reduced growth of established tumours when treated with a combination of dendritic cell therapy and a CD40 agonist. The addition of a CD40 agonist thereby potentiates dendritic cell therapy, leading to a significantly reduced tumour growth compared to untreated mice or mice treated with a CD40 agonist alone or dendritic cell therapy alone.

The CD40 agonist according to the present invention is preferably administered to said patient after said dendritic cells have been administered. However, it is also possible to administer the CD40 agonist simultaneously (i.e. concomitantly) with the loaded dendritic cells.

### The Mesothelioma Cell Lysate

Because differential antigen expression takes place in tumours from different patients, it is not sufficient to provide a lysate derived from only one cell line to a group of patients.

With the present invention this is achieved by preparing a lysate of mesothelioma tumour cells from at least two different cell lines. By using different cell lines multiple antigens are thus present in the lysate, which lysate may be used to load dendritic cells. This way, the chances are reduced that a pancreatic tumour cell in a patient escapes, by down-regulating a specific antigen.

Furthermore, the use of a lysate of said tumour cells is essential for the present invention. Due to the use of this lysate, the different antigens from the different tumour cell lines are directly available to the dendritic antigen presenting cells. Besides the multitude repertoire of antigens, the advantage of using an allogeneic lysate is the off-the-shelf availability and a superior quality compared to autologous lysate.

A key problem associated with the use of autologous tumour cells is that the amount of tumour cells obtained from resected tumour material (either after surgery or through a biopsy) is limited in quantity and quality. Furthermore, the tumour material obtained from patients is, apart from total tumour amount, highly heterogeneous, which makes standardization difficult, and “contaminated” with normal cells (e.g., macrophages, lymphocytes). When this tumour material is then used for the treatment of pancreatic cancer, different outcomes of the phenotype and stimulatory capacity can be expected, with a potential negative impact on efficacy, but also complicating the development of a commercial product. For the reasons set out above, use is made of allogeneic mesothelioma tumour cells for the preparation of the lysate.

In the context of the present invention the term “allogeneic” has its normal scientific meaning and refers to tumour cells which are derived from an individual which is different from the individual to which the lysate resulting from the method according to the present invention shall be later administered. The use of tumour cell lysates from cell lines derived from allogeneic mesothelioma tumour cells provides a more standardized and easier approach, bypassing the need for an individually prepared autologous tumour lysate. It also creates opportunities to select the optimal source, dose and delivery onto dendritic cells or perform manipulations to increase the immunogenicity of the cells. The utilization of a robust and validated large scale manufacturing process also requires fewer product batches for quality control tests such as identity, purity, quantity and sterility/safety testing. A major advantage of the allogeneic approach over autologous is that the tumour cell lines can be selected and optimized, stored in bulk, and manufacturing/quality control timeliness shall not impact on the immediate disease progression of the patient as supply of lysate is off-the-shelf.

In accordance with the present invention the term “necrosis” has its normal scientific meaning and means morphological changes of cells. Necrosis is, inter alia, characterized for example by “leakiness” of the cell membrane, i.e. an increased permeability which also leads to an efflux of the cell's contents and an influx of substances perturbing homeostasis and ion equilibrium of the cell, DNA fragmentation and, finally, to the generation of granular structures originating from collapsed cells, i. e. cellular debris. Typically, necrosis results in the secretion of proteins into the surrounding which, when occurring in vivo, leads to a pro-inflammatory response.

Methods for the determination whether a cell is necrotic are known in the prior art. It is not important which method the person skilled in the art chooses since various methods are known. Necrosis can, e.g., be induced by freeze-thaw cycles, heat treatment, triton X-100, or H202.

Necrotic cells in accordance with the present invention can be determined, e. g., by light-, fluorescence or electron microscopy techniques, using, e. g., the classical staining with trypan blue, whereby the necrotic cells take up the dye and, thus, are stained blue, or distinguish necrotic cells via morphological changes including loss of membrane integrity, disintegration of organelles and/or flocculation of chromatin. Other methods include flow cytometry, e. g., by staining necrotic cells with propidium iodide.

In accordance with the present invention the term “apoptosis” has its normal scientific meaning and means programmed cell death. If cells are apoptotic various changes in the cell occur, such as cell shrinkage, nuclear fragmentation, chromatin condensation, and chromosomal DNA fragmentation.

Apoptotic cells can be determined, e. g. , via flow-cytometric methods, e. g. , attaining with Annexin V-FITC, with the fluorochrome : Flura-red, Quin-2, with 7-amino-actinomycin D (7-AAD), decrease of the accumulation of Rhodamine 123, detection of DNA fragmentation by endonucleases : TUNEL-method (terminal deoxynucleotidyl transferase caused X-UTP nick labelling), via light microscopy by staining with Hoechst 33258 dye, via Western blot analysis, e. g. , by detecting caspase 3 activity by labelling the 89 kDa product with a specific antibody or by detecting the efflux of cytochrome C by labelling with a specific antibody, or via agarose gel DNA-analysis detecting the characteristic DNA-fragmentation by a specific DNA-ladder.

In accordance with the present invention the term “lysing” relates to various methods known in the art for opening/destroying cells. In principle any method that can achieve lysis of the tumour cells may be employed. An appropriate one can be chosen by the person skilled in the art, e. g. opening/destruction of cells can be done enzymatically, chemically or physically. Examples of enzymes and enzyme cocktails that can be used for lysing the tumour cells are proteases, like proteinase K, lipases or glycosidases non-limiting examples for chemicals are ionophores, like nigromycin, detergents, like sodium dodecyl sulfate, acids or bases; and non-limiting examples of physical means are high pressure, like French pressing, osmolarity, temperature, like heat or cold. A preferred way of lysing cells is subjecting the cells to freezing and thawing cycles. Additionally, a method employing an appropriate combination of an enzyme other than the proteolytic enzyme, an acid, a base and the like may also be utilized.

According to the present invention the term “lysate” means an aqueous solution or suspension comprising the cellular proteins and factors produced by lysis of tumour cells. Such a lysate may comprise macromolecules, like DNA, RNA, proteins, peptides, carbohydrates, lipids and the like and/or smaller molecules, like amino acids, sugars, lipid acids and the like, or fractions from the lysed cells. The cellular fragments present in such a lysate may be of smooth or granular structure. Preferably, said aqueous medium is water, physiological saline, or a buffer solution.

The lysate according to the present invention is not limited to lysed necrotic cells. For example, due to the different sensitivity of the treated cells or due to the applied conditions, such as UVB radiation, also lysed apoptotic cells can form or be part of the lysate. It is preferred, however, that the lysate comprises at least 80%, more preferably at least 90%, more preferably at least 95%, most preferably at least 98% lysed necrotic cells. The percentage of lysed necrotic cells can be influenced by the lysing method. Multiple snap-freezing in liquid nitrogen and thawing, for instance, leads to a relative high percentage of necrotic cells, whereas UVB radiation, for instance, leads to a relative high percentage of apoptotic cells. The skilled person is aware of methods for obtaining essentially necrotic cells.

The term lysate as used herein also encompasses preparations or fractions prepared or obtained from the above-mentioned lysates. These fractions can be obtained by methods known to those skilled in the art, e. g. , chromatography, including, e. g., affinity chromatography, ion-exchange chromatography, size-exclusion chromatography, reversed phase-chromatography, and chromatography with other chromatographic material in column or batch methods, other fractionation methods, e. g., filtration methods, e. g., ultrafiltration, dialysis, dialysis and concentration with size-exclusion in centrifugation, centrifugation in density-gradients or step matrices, precipitation, e. g. , affinity precipitations, salting-in or salting-out (ammonium sulfate-precipitation), alcoholic precipitations or other protein chemical, molecular biological, biochemical, immunological, chemical or physical methods to separate above components of the lysates. In a preferred embodiment those fractions which are more immunogenic than others are preferred. Those skilled in the art are able to choose a suitable method and determine its immunogenic potential by referring to the above general explanations and specific explanations in the examples herein, and appropriately modifying or altering those methods, if necessary.

In order to obtain a good immunogenic response it is preferred to use a mixture of allogeneic mesothelioma tumour cells, from at least two mesothelioma tumour cell lines, preferably at least three mesothelioma tumour cell lines, more preferably at least four mesothelioma tumour cell lines, for preparing the lysate. It is particularly preferred to use a mixture of at least five mesothelioma tumour cell lines for preparing the lysate.

Preferably, these at least two, at least three, at least four or at least five mesothelioma tumour cell lines are present in essentially equal cellular amounts at equal concentration preceding lysate preparation. The term “essentially equal cellular amounts” has its conventional meaning and preferably means that each of the cell lines are present in a cell ratio of between 1:2-2:1, relative to one another, more preferably of between 2:3-3:2, more preferably between 3:4-4:3, more preferably between 4:5-5:4, most preferably in a cell ratio of about 1:1.

As an example for five cell lines, the cells could be present in a cell ratio of 3:4:2:4:3, wherein cell line 1 has a ratio of 3:4 to cell line 2, a ratio of 3:2 to cell line 3, a ratio of 3:4 to cell line 4, and a ratio of 1:1 to cell line 5. Cell line 2 has a ratio of 4:3 to cell line 1, a ratio of 2:1 to cell line 3, a ratio of 1:1 to cell line 4, and a ratio of 4:3 to cell line 5. Cell ratios of cell lines 3, 4 and 5 with respect to the others are calculated the same and all fall within the preferred ratios defined above.

Using such mixtures of cell lines as a source of tumour lysate is advantageous in providing a broader antigenic repertoire of tumour associated antigens (wide variety of potential tumour antigens), which will increase the ability of immune responses to recognize and destroy tumour cells because the opportunities to escape immune surveillance by modulation of antigen expression are more limited.

The allogeneic mesothelioma tumour cells, used in the method of the present invention are cultured in for example culture flasks. Due to the fact that these allogeneic cells have the ability to divide unlimitedly with minimal loss of their immunogenic properties, in contrast to non-cancerous cells, they are suitable to use for preparing the lysate. The cell lines that are used for preparing a lysate for use in the present invention are derived from humans.

Presently five human mesothelioma cell lines have been developed that provide particularly good results. These cell lines have been deposited at “Deutsche Sammlung von Mikro-organismen and Zellkulturen” in Germany, hereinafter DSMZ. The cell lines were initially given the following codes and accession numbers: Thorr 01 (deposit No. DSM ACC3191), Thorr 02 (deposit No. DSM ACC3192), Thorr 03 (deposit No. DSM ACC3193), Thorr 04 (deposit No. DSM ACC3194), Thorr 05 (deposit No. DSM ACC3195).The deposit was made pursuant to the terms of the Budapest treaty on the international recognition of the deposit of micro-organisms for purposes of patent procedure.

After the initial deposit, the cell lines were renamed as follows: Thorr 01 was renamed to Thorr 03, Thorr 02 was renamed to Thorr 01, Thorr 03 was renamed to Thorr 02, Thorr 04 was renamed to Thorr 05, and Thorr 05 was renamed to Thorr 06. Throughout the present patent application, the renamed designation are used, i.e.: Thorr 01 (deposit No. DSM ACC3192), Thorr 02 (deposit No. DSM ACC3193), Thorr 03 (deposit No. DSM ACC3191), Thorr 05 (deposit No. DSM ACC3194), Thorr 06 (deposit No. DSM ACC3195).

In a preferred embodiment, therefore, a lysate for use according to the invention is, therefore, provided, wherein the allogeneic mesothelioma tumour cells used are chosen from two or more of the following cell lines Thorr 01 (deposit No. DSM ACC3192), Thorr 02 (deposit No. DSM ACC3193), Thorr 03 (deposit No. DSM ACC3191), Thorr 05 (deposit No. DSM ACC3194), Thorr 06 (deposit No. DSM ACC3195).

Necrosis of the allogeneic mesothelioma tumour cells, can be achieved by methods commonly known in the prior art. However, subjecting the cells to freeze thawing cycles is particularly preferred. Preferably, the cells are made necrotic and lysed by freezing at temperatures below -75 degrees Celsius and thawing at room temperature, particularly snap freezing in liquid nitrogen at temperatures below -170 degrees Celsius and thawing at room temperatures or more, e.g. in a water bath at about 37 degrees Celsius, is most preferred. It is also preferred that said freezing/thawing is repeated for at least 1 time, more preferably for at least 2 times, even more preferred for at least 3 times, particularly preferred for at least 4 times and most preferred for at least 5 times.

Preferably the tumour cells are treated with at least 50 Gy irradiation, preferably at least 100 Gy irradiation. This way it is avoided that any of the tumour cells remains viable. The irradiation treatment can be carried out before or after the tumour cells have been subjected to freezing and thawing.

In one preferred embodiment of a method according to the present invention the lysate comprises at least three mesothelioma cancer cell associated antigens. Preferably, the lysate comprises at least three, more preferably at least five, more preferably at least ten mesothelioma cancer cell associated antigens. In this regard it is further noted that the antigens may be derived from the same protein, i.e. the antigens may be different epitopes from the same protein. However, it is preferred to use antigens which are (or are based) on different tumour cell associated proteins. It is preferred that the at least three, more preferably at least five, more preferably at least ten mesothelioma cancer cell associated antigens are also expressed on pancreatic cancer cells, i.e. these antigens are shared between mesothelioma cancer cells and pancreatic cancer cells, at least in the majority of pancreatic cancer cells to be treated in a patient in need thereof.

It is particular beneficial that the lysate comprises various antigens that cover ideally all tumour cells of a tumour. After all, if a specific tumour cell does not have a specific antigen an immune response will not be triggered against such a cell. If other cells are attacked, but this cell is not, it will have an advantage and will be able to grow further resulting in a further growth of the tumour. The inventors have now been able to establish the most important antigens which can be used to load dendritic cells and target substantially all tumour cells in pancreatic cancer. This approach has allowed the present inventors to formulate lysate which is particularly useful for loading dendritic cells and inducing an immune response to pancreatic cancer cells.

Preferably at least three, more preferably at least five, more preferably at least six of the mesothelioma cancer cell associated antigens are chosen from the group of RAGE1/MOK, Mesothelin, EphA2, Survivin, WT1, MUC1. Further antigens which are of importance within the context of the present invention are RAB38/NY-MEL-1, BING4, MAGE A12, HER-2/Neu, Glypican, LMP2. A mixture of at least three, preferably at least five, more preferably at least six, most preferably at least ten of the mentioned mesothelioma associated antigens is particularly effective against pancreatic cancer when used according to the invention.

In a preferred embodiment, a lysate for use according to the invention is provided, wherein the at least three, preferably at least five, more preferably at least six mesothelioma cancer cell associated antigens are chosen from the group of: RAGE1/MOK, Mesothelin, EphA2, Survivin, WT1, MUC1.

In another preferred embodiment, a lysate for use according to the invention is provided, wherein the at least three, preferably at least five, more preferably at least seven, more preferably at least nine, more preferably at least ten mesothelioma cancer cell associated antigens are chosen from the group of: RAGE1/MOK, Mesothelin, EphA2, Survivin, WT1, MUC1, RAB38/NY-MEL-1, BING4, MAGE A12, HER-2/Neu, Glypican, LMP2.

It has surprisingly been found that many of the antigens, present in mesothelioma cells, used to prepare a lysate of the invention, are shared with pancreatic cancer cells (Table 1). For example, the tumour associated antigen mesothelin, which is abundantly present in the lysate of the invention (further referred to as “PheraLys”), is also present in pancreatic cancers. The presence of mesothelin in pancreatic cancer has led to the initiation of clinical trials worldwide targeting mesothelin for this type of cancer. Combining Listeria Monocytogenes-expressing mesothelin and allogenic pancreatic cancer vaccination GVAX prolonged median survival of advanced pancreatic cancer patients from 3.9 months to 6.1 months (22). However, due to the mono-antigen approach the duration of the response is limited.

TABLE 1
Antigens of interest for pancreatic cancer in PheraLys

| | | Gene | FPKM | | Pancreatic |
|---|---|---|---|---|---|
| Rank^{a} | Antigen | ID | score^{b} | PheraLys | cancer^{c} |
| 3 | Mesothelin | 10232 | 84.25 | ++ | ++ |
| 9 | Survivin | 332 | 38.71 | ++ | ++ |
| 18 | HER-2/neu | 2064 | 16.89 | + | + |
| 21 | MUC1 | 4582 | 13.13 | ++ | ++ |
| 29 | WT1 | 7490 | 10.28 | + | + |
| 30 | KRAS | 3845 | 9.26 | + | + |
| 36 | LY6K | 54742 | 6.84 | +/− | +/− |

^{a}An extensive list (195) of over-expressed, differentiation and cancer germline antigens were checked their frequency within each of the five malignant mesothelioma cell lines that are used to create for PheraLys via RNA sequence analysis and ranked according to their average FPKM score
^{b}FPKM = fragments per kilobase million mapped
^{c}Antigen expression according to www.proteinatlas.org
++ = strong expression, + = medium expression, +/− = expression status differs between samples

In addition to the numerous antigens with relatively high expression, the antigens with relatively low expression may also induce a highly specific T-cell response in the patient. It was, e.g., shown that both dominant and subdominant neoantigens significantly increased the TCR-13 repertoire upon DC vaccination (55). Therefore, all antigens may be of value in the patient and, whereas others have tried a single antigen, or a combination of a few antigens for dendritic cell loading, the magnitude of the number of antigens in PheraLys is clearly an advantage of the current approach.

It has, for instance, been demonstrated that efficacy of mono-antigen treatments is often of short duration in solid tumours (56). Tumours are able to relatively rapidly down regulate that specific antigen after which the treatment becomes ineffective. In contrast, immunotherapy with a multitude of tumour associated antigens decreases the possibility of tumour escape by eliciting a broad immune response and clinical response will be more durable. In one embodiment, the lysate is in the form of a pharmaceutical composition further comprising a pharmaceutically acceptable excipient or carrier, for use in the treatment of pancreatic cancer.

The lysate may also be loaded on dendritic cells ex vivo and formulated into a pharmaceutical composition as will be described in more detail below.

### The Dendritic Cells

The term “dendritic cells” as used herein has its conventional meaning and refers to antigen-presenting cells (also known as accessory cells) of the mammalian immune system, which capture antigens and have the ability to migrate to the lymph nodes and spleen, where they are particularly active in presenting the processed antigen to T cells. The term dendritic cells also encompasses cells which have an activity and function similar to dendritic cells. Dendritic cells can be derived from either lymphoid or mononuclear phagocyte lineages. Such dendritic cells can be found in lymphatic and non-lymphatic tissue. The latter appear to induce a T cell response only when being activated and having migrated to lymphatic tissues.

Dendritic cells are known to be amongst the most potent activators and regulators of immune responses. One important feature is that they are presently the only antigen presenting cells known to stimulate naïve T cells. Immature dendritic cells are characterized by their ability to take-up and process antigens, a function that is dramatically reduced in mature dendritic cells, which in turn exhibit enhanced presentation of processed antigens on their surface, mainly bound to MHC Class I and Class II molecules. Maturation is also associated with upregulation of co-stimulatory molecules (such as CD40, CD80 and CD86), as well as certain other cell surface proteins (e. g. CD83 and DC-Sign). Dendritic cell maturation is also usually associated with enhanced migratory capacity, resulting (in vivo) in migration of dendritic cells to the regional lymph nodes, where the dendritic cells encounter T and B lymphocytes. In a preferred embodiment, the dendritic cells are immature when they are loaded with the lysate, but are mature and activated when administered to a patient in need thereof.

Dendritic cells can be obtained from humans, using methods known to those skilled in the art (57-59). After having obtained the monocytes, these cells are differentiated ex vivo to immature dendritic cells, which are further maturated and activated.

Preferably, the dendritic cells cultured are autologous dendritic cells. The advantage of using autologous dendritic cells is that immune reactions of the patients against these dendritic cells is avoided and that the immunological reaction is triggered against the antigens from the mesothelioma tumour cells, which were present in the lysate.

In a preferred embodiment, the dendritic cells are autologous to the subject having pancreatic cancer. Although using autologous dendritic cells provides many advantages, it may also be advantageous to use allogeneic dendritic cells. One of the major advantages of using allogeneic dendritic cells is that a medicament can be provided to patients that is ready to use. In other words one does not have to differentiate, load and activate the dendritic cells from an individual but one can immediately administer the loaded allogeneic dendritic cells. This saves patient's valuable time. In one preferred embodiment, therefore, the dendritic cells are allogeneic to the subject having pancreatic cancer.

### Loading of the Dendritic Cells with the Mesothelioma Cell Lysate

Dendritic cells or their precursors are differentiated using suitable growth factors and/or cytokines, e. g. GM-CSF and IL-4, the resulting immature dendritic cells are loaded with a lysate for use according to the invention. Immature dendritic cells, loaded with a lysate for use according to the invention, are further maturated to mature dendritic cells. In special cases also mature dendritic cells can be loaded (pulsed) with antigens or immunogens from the lysate.

Preferably, the dendritic cells are loaded with between 1 tumour cell equivalents per 100 dendritic cells to 10 tumour cell equivalents per 1 dendritic cell, preferably between 1 tumour cells per 10 dendritic cells to 1 tumour cell equivalent per 1 dendritic cell. Particularly preferred is about 1 tumour cell equivalent per 3 dendritic cells.

Preferably, a dosage administered to a patient comprises 1*10⁶ to 1*10⁹ loaded dendritic cells, preferably 2*10⁶ to 5*10⁸ loaded dendritic cells, more preferably 1*10⁷ to 1*10⁸ loaded dendritic cells, most preferably about 2.5*10⁷. Most preferably a dose comprises about 2.5*10⁷ dendritic cells loaded with about 1 tumour cell equivalent per 3 dendritic cells.

It is preferred to load the dendritic cells with more than one mesothelioma cancer cell associated antigen. Hence, preferably the composition for loading the dendritic cells comprises at least three, preferably at least five, more preferably at least ten mesothelioma cancer cell associated antigens. In this regard it is further noted that the antigens may be derived from the same protein, i.e. the antigens may be different epitopes from the same protein. However, it is preferred to use antigens which are (or are based) on different tumour cell associated proteins.

In order for the T-cells to be able to attack all tumour cells it is important to make sure that the dendritic cells are loaded with antigens that cover ideally all tumour cells of a tumour. After all, if a specific tumour cell does not have a specific antigen an immune response will not be triggered against such a cell. If other cells are attacked, but this cell is not, it will have an advantage and will be able to grow further resulting in a further growth of the tumour. The inventors have now been able to establish a lysate comprising the most important antigens which can be used to load dendritic cells and target pancreatic cancer. This approach has allowed the present inventors to formulate an antigen composition which is particularly useful for loading dendritic cells and inducing an immune response to pancreatic tumour cells.

The mesothelioma cancer cell associated antigens are preferably chosen from the group of RAGE1/MOK, Mesothelin, EphA2, Survivin, WT1, MUC1. It has been established for the first time that these antigens are able to induce by means of dendritic cell immunotherapy a strong immune reaction against pancreatic tumour cells. Further antigens which are of importance within the context of the present invention are RAB38/NY-MEL-1, BING4, MAGE A12, HER-2/Neu, Glypican, LMP2.

Furthermore, with respect to these tumour cell associated proteins it is noted that as antigens also parts of these proteins (i.e. epitopes thereof) may be used for loading the dendritic cells. In this regard it is further noted that also polypeptides or peptidomimetics of such epitopes may be used for loading the dendritic cells. In one embodiment, the antigen composition comprises only antigens selected from the group of antigens depicted in Table 1. This is advantageous from a regulatory perspective.

In another embodiment the mesothelioma cancer cell associated antigens are obtained from a lysate of allogenic mesothelioma tumour cells from at least two different mesothelioma tumour cell lines, preferably at least three tumour cell lines, more preferably at least four tumour cell lines, most preferably at least five tumour cell lines. The advantage of the use of such a lysate is that many tumour associated antigens will be present in the lysate and that the dendritic cells are loaded with a considerable number of antigens, reducing the chances that a tumour cell will not be recognized and escapes the immune reaction.

The mesothelioma tumour cell lines used for preparing such a lysate are preferably chosen from Thorr 01 (deposit No. DSM ACC3192), Thorr 02 (deposit No. DSM ACC3193), Thorr 03 (deposit No. DSM ACC3191), Thorr 05 (deposit No. DSM ACC3194), Thorr 06 (deposit No. DSM ACC3195).

Said lysate is prepared from between 10*10⁶ and 200*10⁶ tumour cells/ml, preferably between 20*10⁶ and 100*10⁶, more preferably from between 30*10⁶ and 75*10⁶, more preferably from between 40*10⁶ and 60*10⁶ most preferably from about 50*10⁶ tumour cells/ml. Hence, the lysate according to the present invention comprises an equivalent of between 10*10⁶ and 200*10⁶, preferably of between 20*10⁶ and 100*10⁶, more preferably of between 30*10⁶ and 75*10⁶, more preferably of between 40*10⁶ and 60*10⁶, most preferably an equivalent of about 50*10⁶ tumour cells per ml. With equivalent in this context is meant the amount of tumour cells present in solution before lysis, as after lysis only fragments of cells are present.

It has further been found that the total protein content of the lysate for use according to the invention is of relevance, as this is directly related to the number of tumour cells used for preparing the composition. If the amount of protein (i.e. antigen) is too low the loading of dendritic cells will be poor and the induced immune response will be limited. If the protein concentration is too high, interactions between the different proteins will occur, making the antigens less available for absorption by the dendritic cells and causing stability problems. Hence, the total amount of protein in the antigen composition is preferably between 5 and 25 mg protein per ml, more preferably between 6 and 20 mg protein per ml, more preferably between 7 and 15 mg, most preferably between 7.9 and 11.8 mg protein per ml.

It is further preferred that only fragmented DNA is present in the lysate. First, the lysate is preferably subjected to freeze-thawing cycles (decreases the size of DNA) and preferably irradiated to an extremely high dose of 50 Gy, preferably 100 Gy of irradiation that leads to double strand breaks that cannot be repaired and thus leads to distorted and illegible information (reduction of the oncogenic and infectious risk of residual DNA). Further, dendritic cells are preferably purified from non-incorporated lysate constituents by density gradient centrifugation, thereby removing residual small DNA-fragments. After removal of lysate from the dendritic cells, dendritic cells are preferably incubated ex vivo for at least 12 hours, preferably at least 24 hours, more preferably at least 48 hours before purification, thereby allowing free floating nucleic acid (RNA/DNA) to be degraded by natural nucleases. These measures lead to little complications in the downstream processing of both the lysate and the pharmaceutical composition, including the dendritic cells (no viscosity or complex formation, indicating the absence of sizeable DNA fragments). Although the DNA present in the lysate and/or the pharmaceutical composition is considered as cellular contaminant rather than a risk factor by the WHO Expert Committee on Biological Standardization, they set a dose limit of 10 ng/dose.

Therefore, the pharmaceutical composition according to the present invention preferably comprises less than 10 ng free DNA per dose, preferably less than 100 pg, more preferably less than 1 pg, most preferably less than 0.01 pg free DNA per dose.

In a preferred embodiment, a lysate for use according to the invention is provided, wherein the lysate is loaded onto autologous dendritic cells before administering the lysate to a patient. Preferably, the dendritic cells are loaded with between 1 tumour cell equivalents per 100 dendritic cells to 10 tumour cell equivalents per 1 dendritic cell, more preferably between 1 tumour cell equivalent per 100 dendritic cells to 1 tumour cell equivalent per 1 dendritic cell, most preferably with about 3 dendritic cells to 1 tumour cell equivalent.

In order to induce a sufficiently large immune response it is advantageous to administer a patient in need thereof with between 1*10⁶ to 1*10⁹ loaded dendritic cells per administration, preferably 2*10⁶ to 5*10⁸ loaded dendritic cells, more preferably 1*10⁷ to 1*10⁸ loaded dendritic cells, most preferably about 2.5*10⁷ dendritic cells per administration.

The dendritic cells used may be autologous or allogenic. However, it is particularly preferred to use autologous dendritic cells. MHC class II molecules expressed on these autologous dendritic cells display peptides to the TCR expressed on T cells present in the treated patient. The ability of the TCR to discriminate foreign peptides from self-peptides presented by “self” MHC molecules is a requirement of an effective adaptive immune response. Use of allogenic dendritic cells, injected intra-tumoural has also been described, but it is unlikely that such allogeneic dendritic cells present the tumour antigens directly to the patient's T cells (60). Without being bound to theory it is believed that such allogeneic dendritic cells, when injected at the site of the tumour, may effectively recruit other immune cells to the site, e.g., NK cells, which ultimately kill the allogeneic dendritic cells, thereby providing both the tumour antigens and a “danger signal” to intra-tumoural autologous dendritic cells that than induce a specific (T-cell) immune response towards the tumour antigens. In one preferred embodiment, therefore, a dendritic cell of the invention is allogeneic to the patient receiving it, wherein, preferably, the dendritic cell is administered intra-tumourally. Preferably, the lysate is provided as an off-the-shelve product, which can be used to load dendritic cells obtained from a patient suffering from pancreatic cancer. After loading and appropriate formulation for intravenous and/or intradermal administration, the loaded dendritic cells are administered to the patient.

### The Pharmaceutical Composition

The lysate as such and the loaded dendritic cells may be formulated as a pharmaceutical composition or kit. The skilled person will be able to prepare on the basis of his common general knowledge suitable pharmaceutical compositions.

The pharmaceutical composition according to the present invention may comprise or may be administered with a physiologically acceptable carrier to a patient, as described herein. The term “carrier” refers to a diluent, adjuvant, excipient, or vehicle with which the therapeutic is administered. Such pharmaceutical carriers can be sterile liquids, such as water and buffers.

Examples of suitable pharmaceutical carriers are described in “Remington's Pharmaceutical Sciences” by E. W. Martin. Such compositions may comprise a therapeutically effective amount of the cell lysate, or loaded dendritic cells preferably in purified form, together with a suitable amount of carrier so as to provide the form for proper administration to the patient. The formulation should suit the mode of administration.

In an embodiment, the compositions are in a water-soluble form, such as pharmaceutical acceptable salts, which is meant to include both acid and base addition salts.

The compositions can be prepared in various forms, such as injection solutions, tablets, pills, suppositories, capsules, suspensions, and the like.

Pharmaceutical grade organic or inorganic carriers and/or diluents suitable for oral and topical use can be used to make up compositions containing the therapeutically active compounds. Diluents known in the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The compositions may also include one or more of the following: carrier proteins such as serum albumin; buffers; fillers such as microcrystalline cellulose, lactose, corn and other starches; binding agents; sweeteners and other flavouring agents; colouring agents; and polyethylene glycol. Additives are well known in the art, and are used in a variety of formulations.

The pharmaceutical composition may be formulated in accordance with routine procedures as a pharmaceutical composition adapted for intravenous and/or intradermal administration to human beings. Typically, compositions for intravenous and/or intradermal administration are solutions in sterile isotonic aqueous buffer.

The present invention will be elucidated further by means of the following non-limiting examples.

### EXAMPLES

### Example 1

### Description of PheraLys Manufacturing Process

PheraLys is considered a highly heterogeneous source of Tumour Associated Antigens (TAA) due to the inclusion of five highly heterogeneous MPM tumour cell lines.

Cell lines, named Thorr 01, Thorr 02, Thorr 03, Thorr 05 and Thorr06 (Thorr is the abbreviation for Thoracic Oncology Research Rotterdam) from 5 different patients with MM were selected for PheraLys preparation. These cell lines are deposited for patent purposes according to the Budapest Treaty at the Leibniz Institute DSMC-Collection of Microorganisms and Cell Cultures (DSMZ): Thorr 01 (deposit No. DSM ACC3192), Thorr 02 (deposit No. DSM ACC3193), Thorr 03 (deposit No. DSM ACC3191), Thorr 05 (deposit No. DSM ACC3194), Thorr 06 (deposit No. DSM ACC3195).

Individual Thorr cell lines are brought into culture and are incubated in a humidified atmosphere of 5% CO2, 95% air at 37° C. overnight followed by a medium exchange and a

PBS wash the following day. The cells are washed and expanded in fresh medium until a sufficient number of cells for each individual Thorr cell line are obtained. Cells are washed extensively with PBS, counted and stored at a concentration of 50×10⁶ cells per ml in PBS at <−70° C. in a controlled environment until further use.

Equal cellular amounts of the different cell lines are mixed and stored at <−70° C. For preparation of the lysate, the intermediate product is thawed and aliquoted in 50 ml tubes, containing 30 ml of cell suspension. These 50 ml tubes are freeze-thawed 5 times by snap-freezing with liquid nitrogen. Thereafter, the 50 ml tubes are irradiated with 100 Gy by gamma irradiation with a Radioactive 137Cesium irradiation source (Cis Bio International). As of this point there are no more tumour cells present in the finalized lysate, therefore concentration is mentioned in Tumour Cell Equivalent (TCE). 50*10⁶ TCE equals the content of 50*10⁶ tumour cells.

### Example 2

### Tumour Associated Antigen Expression in Thorr Cell Lines

The five tumour cell lines have been characterized by RNA sequencing with Affymetrix expression arrays. The expression profiles of the cell lines were evaluated against a list of 195 known antigens. This list of 195 antigens encompasses all differentiation/overexpressed antigens that are published in literature either as targets or prognostic markers. Furthermore it includes all cancer germline antigens that are currently listed as cancer-specific targets in the cancer/testis antigen database (www.cta.lncc.br). Cancer germline antigens are of specific interest as these have a bigger chance to trigger powerful immune responses since they are only expressed by cancer cells and not by healthy tissue.

FPKM (fragments per kilobase per million) approximates the relative abundance of transcripts in terms of fragments observed from an RNA-sequence experiment. Longer genes will have more fragments than shorter genes if transcript expression is the same. This is adjusted by dividing the FPM by the length of a gene, resulting in the metric fragments per kilobase of transcript per million mapped reads (FPKM).

The results show that the TAA of interest are heterogeneously expressed by the different

Thorr cell lines (Table 2). This exemplifies the potential of the 5 selected Thorr cell lines to act as a broad, clinically relevant, TAA source.

TABLE 2
Most relevant antigens present in the model cell lines (RNA sequencing results)

| | | Amount of | Amount of | Amount of | Amount of | Amount of |
|---|---|---|---|---|---|---|
| | | antigen | antigen | antigen | antigen | antigen |
| | | expressed | expressed | expressed | expressed | expressed |
| | Gene | in Thorr | in Thorr | in Thorr | in Thorr | in Thorr |
| Antigen | ID | 01* | 02* | 03* | 05* | 06* |
| RAGE-1/MOK | 5891 | 1.91 | 10.7 | 50.73 | 310.14 | 48.91 |
| Mesothelin | 10232 | 42.89 | 50.9 | 69.36 | 143.6 | 114.49 |
| EphA2 | 1969 | 32.65 | 97.77 | 24.82 | 62.6 | 162.78 |
| Survivin | 332 | 46.83 | 39.53 | 49.07 | 38.28 | 19.86 |
| WT1 | 7490 | 6.47 | 29.49 | 0.45 | 0.28 | 14.71 |
| MUC1 | 4582 | 10.31 | 12.91 | 11.11 | 18.72 | 12.58 |
| RAB38/NY-MEL-1 | 23682 | 3.21 | 0.27 | 0.48 | 0 | 0.07 |
| BING-4 | 9277 | 20.22 | 17.65 | 37.07 | 24.34 | 34.28 |
| MAGE-A12 | 4111 | 0 | 0 | 51.8 | 0 | 0 |
| HER-2/neu | 2064 | 18.69 | 11.54 | 14.73 | 16.14 | 23.36 |
| glypican-1 | 2817 | 128.93 | 29.62 | 43.47 | 92.31 | 66.31 |
| LMP2 | 5698 | 29.77 | 148.59 | 4.14 | 111.89 | 158.2 |

*FPKM values (fragments per kilobase of exons per million fragments mapped).

### Example 3

### Immune Response Directed Against Pancreatic Tumour by Treatment with DCs Loaded with Either Autologous Pancreatic or Allogeneic Mesothelioma Lysate

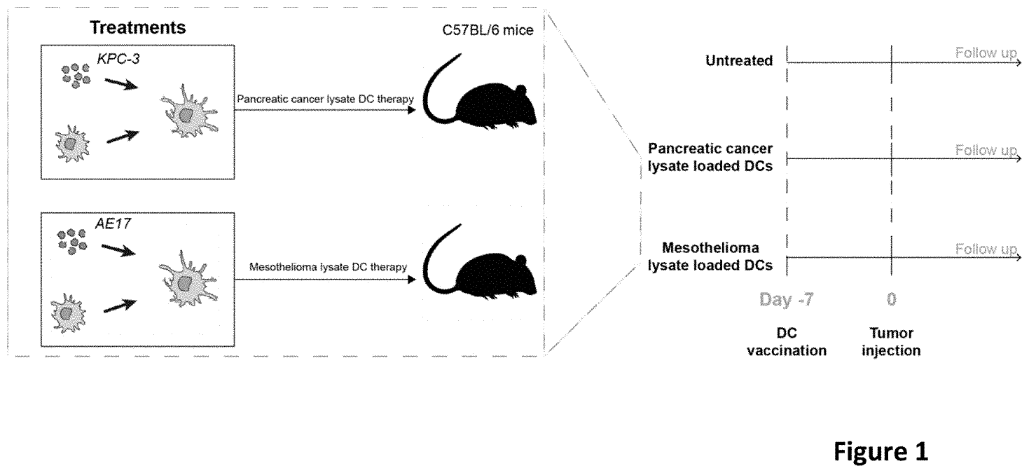

Immunocompetent C57bl/6 mice were treated with DC-vaccines consisting of monocyte-derived DCs loaded with either pancreatic cancer lysate (KPC-3) or with mesothelioma lysate (AE17). Loading was comparable to the human situation, i.e. 1 tumour cell equivalent per 3 DCs. An untreated group was also included. Subsequently, a pancreatic tumour was induced by subcutaneous injection with 100,000 cells of the pancreatic cancer KPC-3 cell line and tumour growth was followed (see for schematic setup: FIG. 1). This experimental set-up corresponds to the situation of pancreatic cancer patients after surgery, with only micro-metastases left.

In this preclinical setting, 2×10⁶ DCs were injected subcutaneously and 1×10⁶ DCs intravenously seven days before tumour implantation. Since pancreatic cancer patients are intended to receive vaccination post-surgery, having no clinical signs of established tumour nor presence of desmoplastic stroma distinctive for established pancreatic cancer, vaccination prior to tumour establishment in our mouse model closely resembles the clinical setting. By treating mice before the establishment of macroscopic tumour formation and desmoplasia we mimic resected patients with potential presence of micrometastatic disease. DCs were stimulated overnight with CpG and loaded with either mesothelioma lysate (AE17 cell line; prof. Nelsons, Curtin University, Perth, Australia) or pancreatic cancer lysate (KPC-3). DCs were generated as previously described (54).

The systemic immune response was monitored 4 and 11 days following DC vaccination (interim analysis). At end-stage disease (27 days following DC vaccination), T cell phenotype (including activation, proliferation and exhaustion status) was analyzed in tumour, spleen and peripheral blood (end-stage analysis).

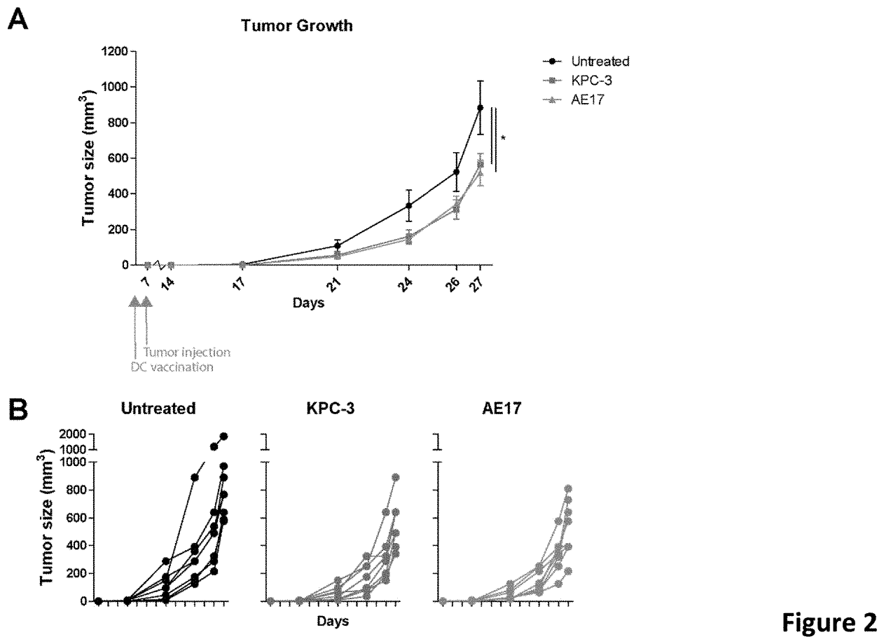

Tumour growth was significantly delayed in treated animals compared to untreated animals. The relative delay in tumour growth and tumour sizes at the different time points were comparable in the treated animals irrespective of the type of loading of the DCs, indicating that DC therapy with mesothelioma cell lysate is as effective as DC therapy with autologous pancreatic cell lysate (FIG. 2).

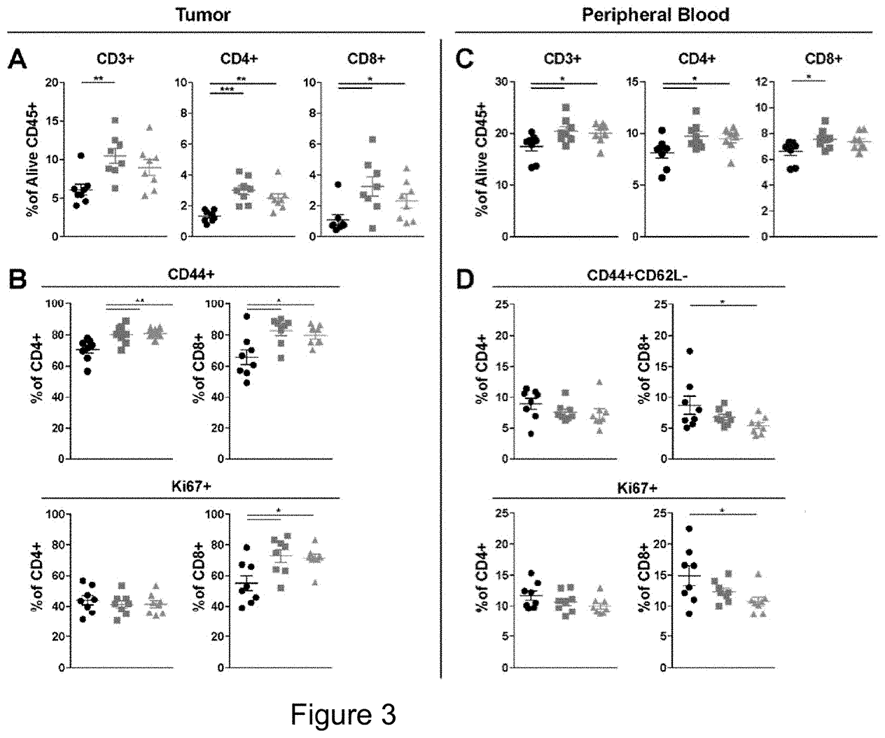

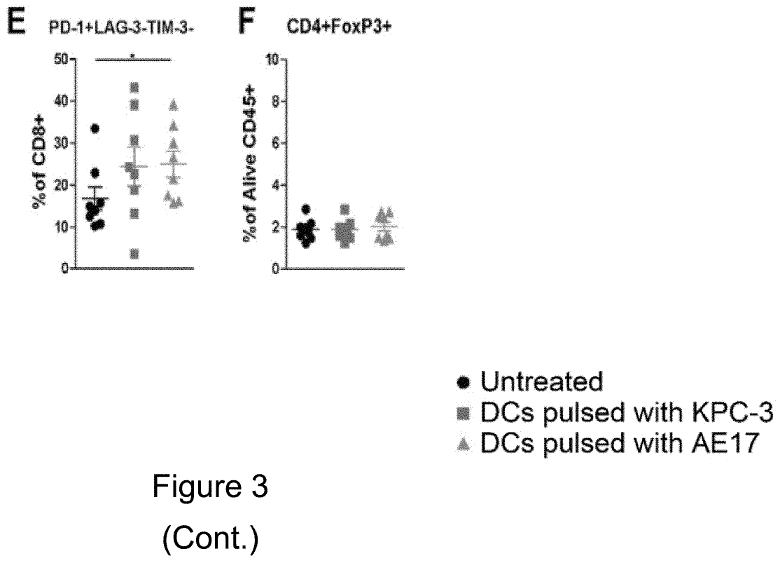

Delay of tumour growth was accompanied by increased frequencies of tumour infiltrating lymphocytes (TILs) in both groups of DC treated mice compared to untreated mice (FIG. 3A). Also, CD44 expression was higher on both CD4+ and CD8+ TI Ls in treated mice indicating a more prominent effector memory T cell phenotype. The proliferation marker Ki67 was also higher on CD8+ TI Ls in treated mice compared to untreated mice (FIG. 3B). In addition, higher frequencies of PD-1+ LAG-3−0 TIM-3− CD8+ TI Ls were observed in treated mice, although with significant variation. This phenotype is associated with truly activated non-exhausted T cells needed for a robust anti-tumour response (FIG. 3E).

There was no increase in suppressive intra-tumoural CD4+FoxP3+ Tregs after DC therapy (FIG. 3F), which further substantiates an effective anti-tumour CD8+ T-cell response. In peripheral blood, increased frequencies of T-cell subsets could be observed as early as four days after DC treatment. The increased frequencies of T-cells in peripheral blood and spleen (not shown) were still present 27 days after treatment, whereas the earlier observed enhanced values of CD44+CD62L− subsets and the Ki67 marker were restored to untreated baseline (FIG. 3C, D).

To demonstrate the induction of a tumour-specific T-cell response, splenocytes were isolated on the day of sacrifice of the mice of Experiment I. CD8⁺ MACS-purified splenocytes were in vitro stimulated with pancreatic tumour cells (KPC-3).

Upon stimulation with pancreatic tumour cells increased frequencies of various activation and degranulation markers were expressed by CD8⁺ T-cells of treated mice compared to untreated mice.

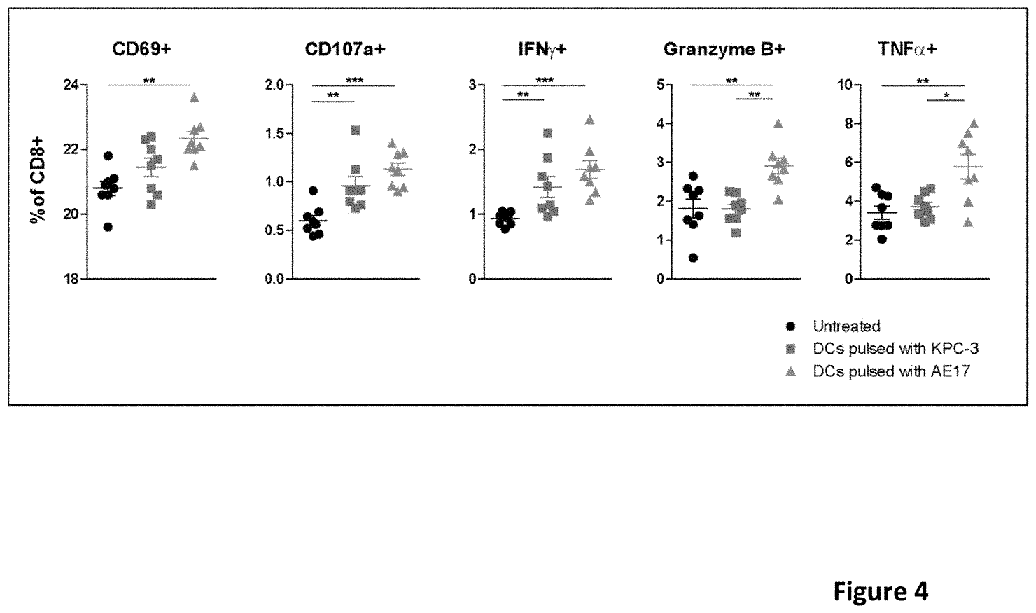

Interferon-γ (IFNγ) and tumour necrosis factor a (TNFα) production was assessed by intracellular cytokine staining, and expressions of CD107a, CD69 and granzyme B were also assessed by flow cytometry. Notably, the frequencies of IFNγ⁺ and CD107a⁺ expressing CD8⁺ T-cells were increased upon stimulation with tumour cells in all treated mice in comparison to untreated mice. In the case of CD69, granzyme B and TNFα, only higher frequencies could be observed in mice treated with mesothelioma-pulsed DCs (FIG. 4).

### Example 4

### Loading of DCs with (Shared) Tumour Associated Antigens Prerequisite for an Effective Anti-Tumour Response

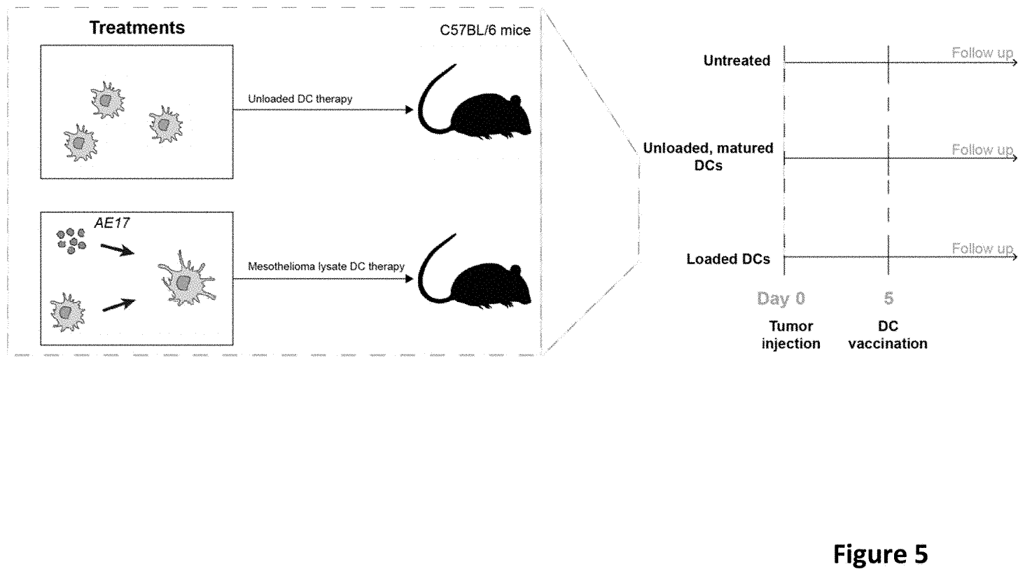

It was investigated whether delayed tumour growth is dependent on the induction of a tumour-specific immune response induced by DCs loaded with tumour associated antigens shared between mesothelioma and pancreatic cancer cell lysate or by the administration of matured DCs as such. To this end, KPC-3 C57BI/6 mice were treated with either unloaded (i.e. in the absence of tumour lysate) but matured DCs (stimulated with CpG) or DCs that were matured and loaded with the mesothelioma AE17 lysate (see for schematic setup: FIG. 5).

Unloaded, but matured DCs (referred to as unloaded DCs or DCs only) are not deliberately loaded with tumour-specific antigens. However, matured DCs will present peptides with which they came into contact and DCs will never express MHC molecules without bound peptide in the MHC groove. In this experiment DCs will have taken up peptides during the culturing process. These peptides/antigens will most likely not overlap with tumour associated antigens.

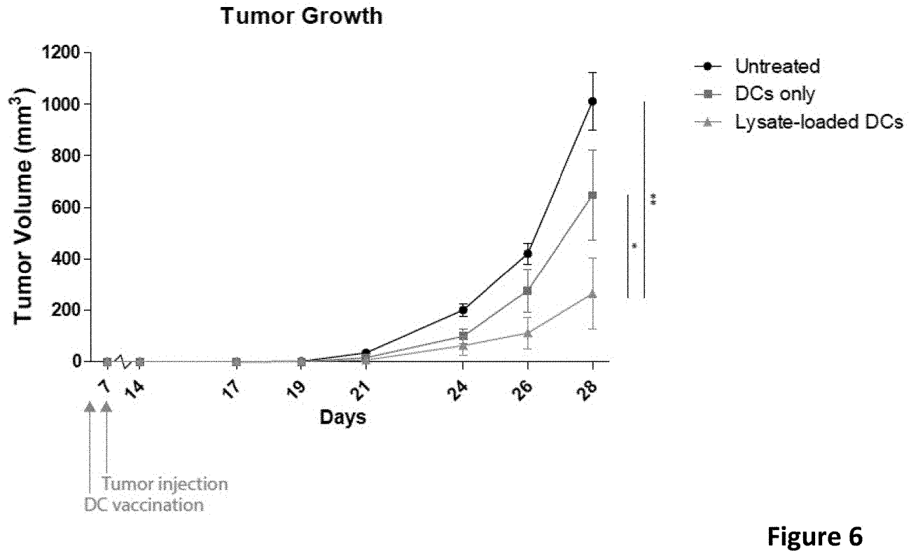

Mice treated with mesothelioma lysate loaded DCs had a significant delayed tumour growth, indicating that loading DCs with mesothelioma lysate induces a tumour-specific immune response directed against the pancreatic tumour (FIG. 6).

### Example 5

### Induction of Pancreas Tumour-Specific Immune Response

To monitor whether mesothelioma lysate loaded DCs induce a pancreas tumour-specific immune response, splenocytes and tumours from treated and untreated tumour-bearing mice from Example 4 were isolated on the day of sacrifice. Bone marrow was harvested from wild type non-tumour bearing mice for the culture of mature DCs.

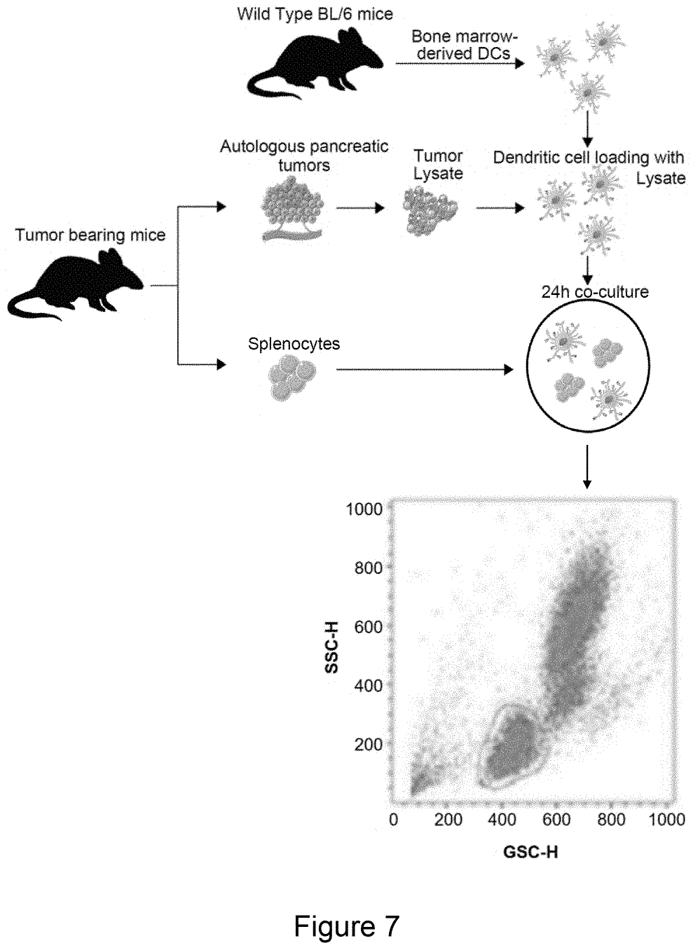

DCs were cultured from mouse bone marrow with GM-CSF and loaded with autologous pancreas tumour lysate or with healthy lung lysate as a control. Autologous pancreatic lysate and healthy lung lysate were made from snap frozen end stage tumours or lung tissue, respectively, by bead mediated homogenisation. DCs loaded with autologous pancreatic tumour- or control lung lysate were co-cultured with thawed splenocytes for 24 hours. A schematic overview of this (potency) assay is depicted in FIG. 7.

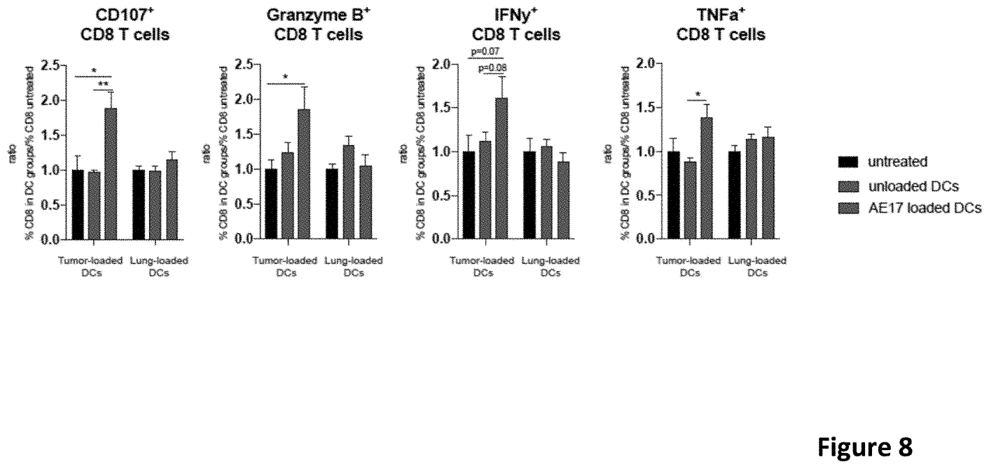

Upon co-culture of autologous pancreatic tumour lysate loaded DCs with splenocytes from mice treated with mesothelioma loaded DCs, we found an increased expression of the cytotoxic markers CD107, Granzyme B, and pro-inflammatory cytokines IFNγ and TNFα in CD8+ T-cells, as compared to splenocytes from untreated mice or from mice treated with unloaded DCs (FIG. 8).

The increase in these cytotoxic markers and pro-inflammatory cytokines was not observed when DCs loaded with control lung lysate were co-cultured with splenocytes from treated or untreated mice.

### Example 6

### Description of Manufacturing Process of MesoPher Drug Substance for Clinical Use (Dendritic Cells, Loaded with a Tumour Lysate)

The apheresis product is the cellular starting material, it is generated by standard 9L leukapheresis procedure to collect mononuclear cells using an apheresis unit according to hospital procedures. After the procedure, the product is transferred to the cleanroom and prepared for CliniMACS procedure by labeling with CD14+ Microbeads. The CD14+ monocyte cell product is transferred to 200 ml conical tubes, centrifuged, and resuspended in X-VIVO15 medium supplemented with 2% Human serum/HS(=culture medium) into a final concentration of 100*10⁶ /30 ml. This cell suspension is seeded into 225 cm² culture flasks, 30 ml per flask. The flasks are incubated overnight in a 37° C., 5% CO₂ incubator. The remaining cells are cryopreserved in 10% DMSO.

At day 2, 15 ml of culture medium is replaced with 15 ml fresh culture medium supplemented with cytokines GM-CSF and IL-4 for each culture flask. The final concentration of the cytokines is 800 IU/ml GM-CSF and 500 IU/ml IL-4. The monocytes are cultured at 37° C., 5% CO₂ for 4 days.

At day 5, cells are harvested from the flasks into 200 ml tubes and centrifuged. The cell product is diluted to 0.5×10⁶/ml using culture medium in an end volume of maximum 840 ml (420*10⁶ DC) and minimum 200 ml (100*10⁶ DC). This suspension is supplemented with 800 UI/ml GM-CSF, 500 IU/ml IL-4, 1:3 TCE PheraLys product/DC (TCE: tumour cell equivalent), and 10 ug/ml endotoxin-free Keyhole Limpet Hemocyanin (KLH). This cell suspension is plated into 6-wells plates. The 6-well tissue culture plates are incubated for 2 additional days in a 37° C., 5% CO₂ incubator.

At day 8, DC are matured through the addition of fresh culture medium supplemented with maturation factors to a final concentration of 5 ng/ml IL-1β, 15 ng/ml IL-6, 20 ng/ml TNF-α and 10 μg/ml PGE2. The 6-well tissue culture plates are incubated for 2 additional days in a 37° C., 5% CO₂ incubator.

At day 10, the mature DC are harvested and centrifuged. After centrifugation, culture supernatant is collected separately. Cells are resuspended and pooled in 50 ml PBS. On this suspension a density gradient centrifugation (Lymphoprep) step is performed in 2×50 ml tubes to remove excess PheraLys. Cells are collected from the interface of the gradient (the DC) and washed in PBS by centrifugation. End volume of this suspension is 50 ml in a 50 ml tube. Total cell numbers are defined by a cell counting.

The cell suspension generated in Step 10 is defined as MesoPher Drug Substance (DS).

### Example 7

### Clinical Use of a Lysate or Pharmaceutical composition According to the Invention for the Treatment of Pancreatic Cancer

A phase II study with MesoPher in patients with pancreatic cancer is enrolling. The study synopsis is as follows:

Objectives: To investigate feasibility, safety and toxicity as well as the induced immune response upon vaccination with an allogeneic tumour cell lysate loaded onto autologous dendritic cells in resected pancreatic cancer patients who received standard of care treatment.
Study design: An open-label, single centre phase II study
Study Population: Patients older than 18 years with surgically resected pancreatic cancer who received standard of care treatment
Sample size: 10 patients

Investigational treatment:

Formulation: MesoPher: autologous monocyte-derived dendritic cells loaded with PheraLys
Dose: 25 million loaded DCs
Route of administration: ⅓ intradermal injection in the forearm and ⅔ intravenously
Number of doses: Five vaccinations in total.
Schedule of doses: 3 biweekly doses and 2 additional gifts (3 and 6 months after last dose)
Inclusion criteria:
Surgically resected pancreatic cancer.
Completed post-operative standard treatment. Standard of care treatment includes the choice of adjuvant chemotherapy. Patients who did not complete adjuvant chemotherapy due to toxicity or who are not able to start standard of care due to specific reasons are allowed to participate in the study after approval of the coordinating investigator.
No disease activity as assessed by radiological imaging.
Patients must be at least 18 years old and must be able to give written informed consent.
Patients must be ambulatory (WHO-ECOG performance status 0.1 or 2) and in stable medical condition.
Patients must have normal organ function and adequate bone marrow reserve: absolute neutrophil count >1.0×109/l, platelet count >100×109/l, and Hb >6.0 mmol/l (as determined during screening).
Positive DTH skin test (induration >2 mm after 48 hrs) against at least one positive control antigen tetanus toxoid (see section 8.3 for DTH skin test procedure).
Women of childbearing potential must have a negative serum pregnancy test at screening and a negative urine pregnancy test just prior to the first study drug administration on Day 1, and must be willing to use an effective contraceptive method (intrauterine devices, hormonal contraceptives, contraceptive pill, implants, transdermal patches, hormonal vaginal devices, infusions with prolonged release) or true abstinence (when this is in line with the preferred and usual lifestyle)* during the study and for at least 12 months after the last study drug administration. *True abstinence is acceptable when this is in line with the preferred and usual lifestyle of the subject. Periodic abstinence (such as calendar, ovulation, symptothermal, postovulation methods) and withdrawal are not acceptable methods of contraception. Men must be willing to use an effective contraceptive method (e.g. condom, vasectomy) during the study and for at least 12 months after the last study drug administration.
Ability to return to the hospital for adequate follow-up as required by this protocol.
Written informed consent according to ICH-GCP.

Exclusion criteria:
Medical or psychological impediment to probable compliance with the protocol.
Current or previous treatment with immunotherapeutic agents.
Current use of steroids (or other immunosuppressive agents). Patients must have had 6 weeks of discontinuation and must stop any such treatment during the time of the study. Prophylactic usage of dexamethasone during chemotherapy is excluded from this 6 weeks interval.
Prior malignancy except adequately treated basal cell or squamous cell skin cancer, superficial or in-situ cancer of the bladder or other cancer for which the patient has been disease-free for five years.
Serious concomitant disease, or active infections.
History of autoimmune disease or organ allografts (or with active acute or chronic infection, including HIV and viral hepatitis).
Serious intercurrent chronic or acute illness such as pulmonary disease (asthma or COPD), cardiac disease (NYHA class III or IV), hepatic disease or other illness considered by the study coordinator to constitute an unwarranted high risk for investigational DC treatment.
Known allergy to shell fish (may contain keyhole limpet hemocyanin (KLH)).
Pregnant or lactating women.
Inadequate peripheral vein access to perform leukapheresis.
Concomitant participation in another clinical intervention trial (except participation in a biobank study).
An organic brain syndrome or other significant psychiatric abnormality which would compromise the ability to give informed consent, and preclude participation in the full protocol and follow-up.
Absence of assurance of compliance with the protocol. Lack of availability for follow-up assessment.

### Example 8

### CD40 Agonist Potentiates Mesothelioma Lysate-Pulsed DC Immunotherapy

### Delayed Tumour Growth was Observed in Mice Treated with CD40 and DC Vaccination

Immunocompetent C57bl/6 mice were subcutaneously injected in the right flank with 1*10⁵ pancreatic tumour cells. Mice were treated with DC vaccines consisting of monocyte-derived DCs loaded with mesothelioma lysate (AE17), CD40 agonistic monoclonal antibody (FGK45,

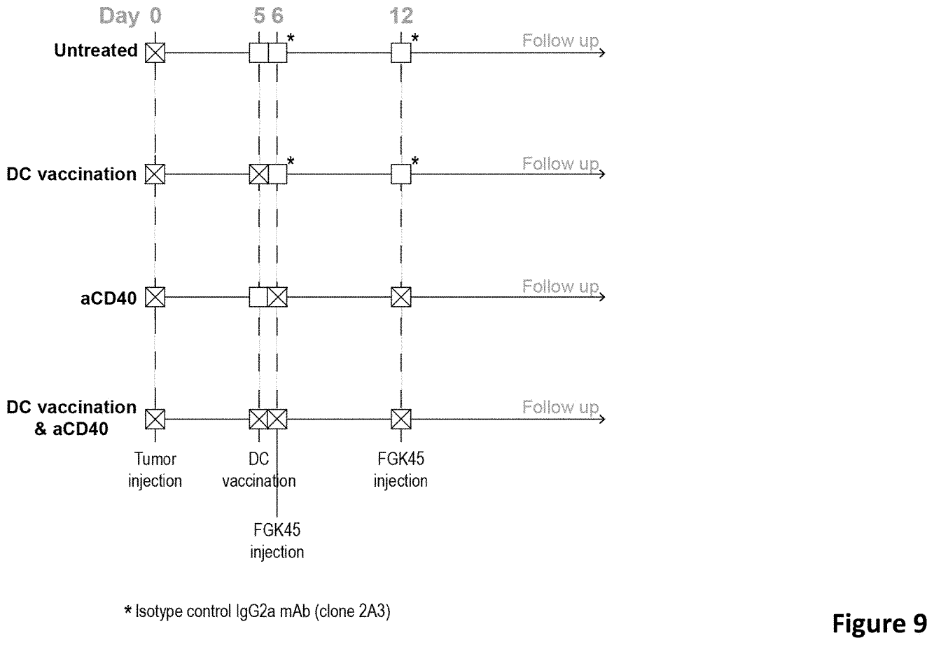

Bio X Cell), or both. 2*10⁶ DCs were injected subcutaneously and 1*10⁶ DCs intravenously at day 5 post-tumour injection. Also, 100 μg of CD40 agonistic monoclonal antibody or its isotype (clone 2A3, Bio X Cell) was injected on day 6 and day 12. Monitoring of mice included measuring tumour sizes 2-3 times a week until the tumour reached 1000 mm³ (FIG. 9).

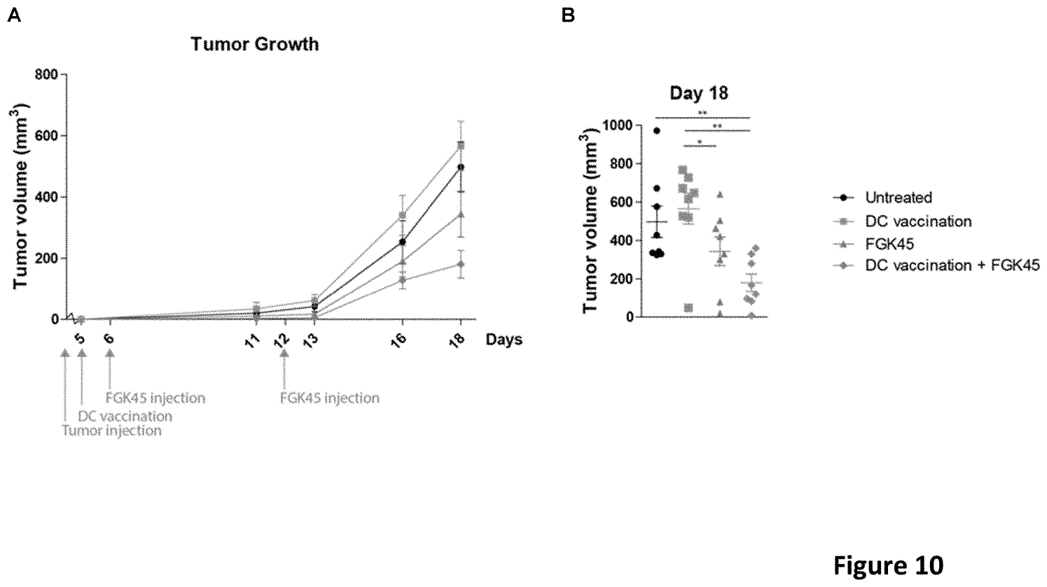

Mice treated with DC vaccination and the CD40 agonistic monoclonal antibody had significantly delayed tumour growth compared to untreated mice. This delay in tumour growth was not observed in mice treated with DC monotherapy or CD40 agonistic monotherapy alone (FIG. 10).

### DC Vaccination has Pronounced Effects on the CD4+ T cell Compartment

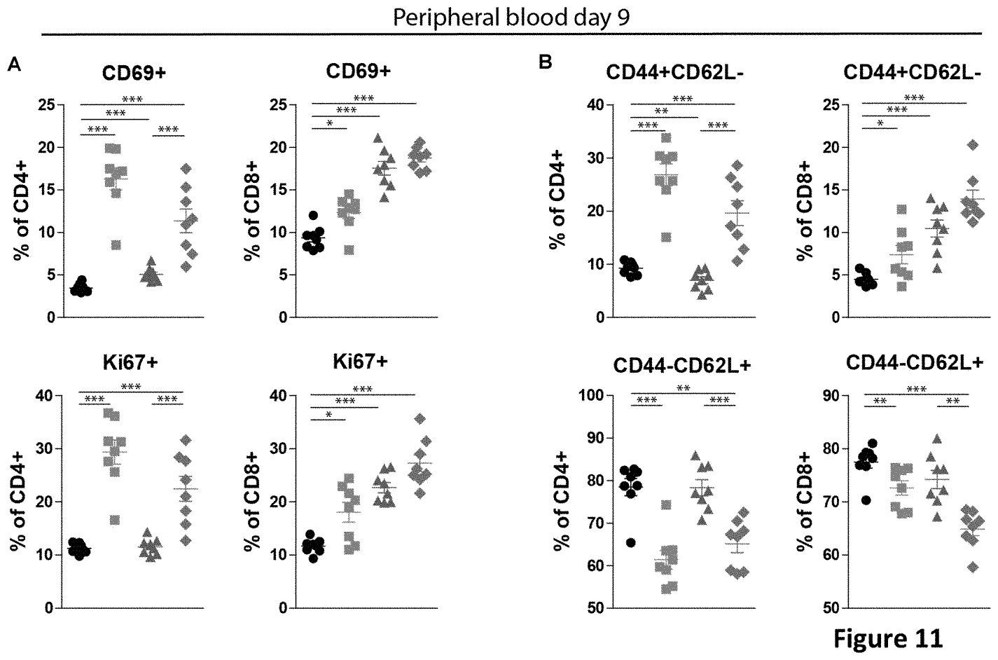

FACS analysis was performed on blood samples of all mice on day 9 post-tumour injection. Increased frequencies of the activation marker CD69 and proliferation marker Ki67 on both CD4 and CD8 T cells was seen in mice treated with both DC vaccination and CD40 agonist treatment compared to untreated mice. There is a striking difference in activation and proliferation of CD4⁺ T cells between mice treated with CD40 agonistic monotherapy or upon combination with DC therapy (FIG. 11A). Similar effects were observed in the T cell effector memory compartment, characterized as CD44⁺ CD62L⁻ (FIG. 11B).

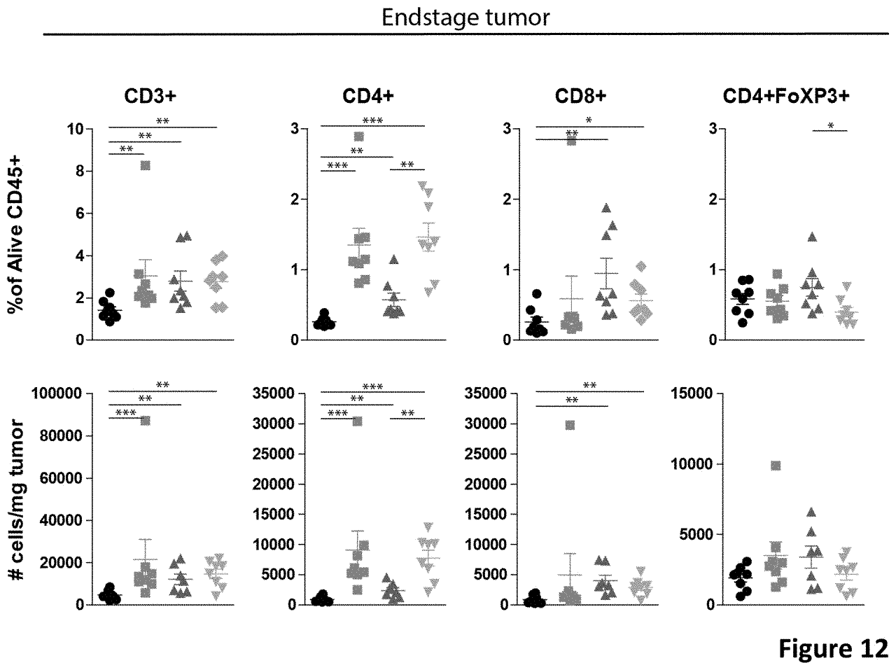

Tumours were isolated and analysed by flow cytometry at end stage disease. Increased frequencies of tumour infiltrating lymphocytes can be observed in all treatment groups as compared to untreated mice. Increased CD4⁺ T cell numbers was more pronounced in the treatment groups that received DC vaccination. T regulatory cell frequencies and numbers were not increased after CD40 agonistic treatment, DC therapy or combination therapy as compared to untreated mice (FIG. 12).

### Treatment of Larger Tumours (10 Days)

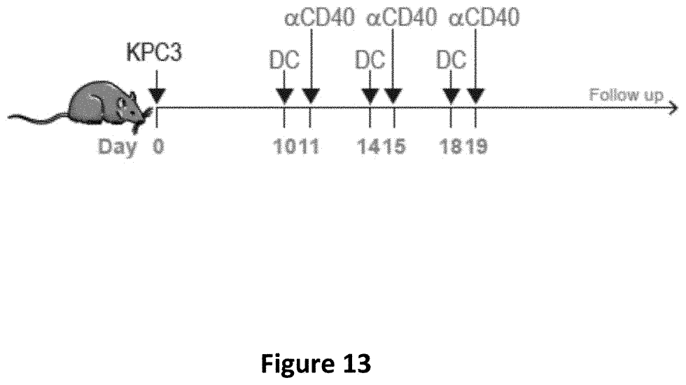

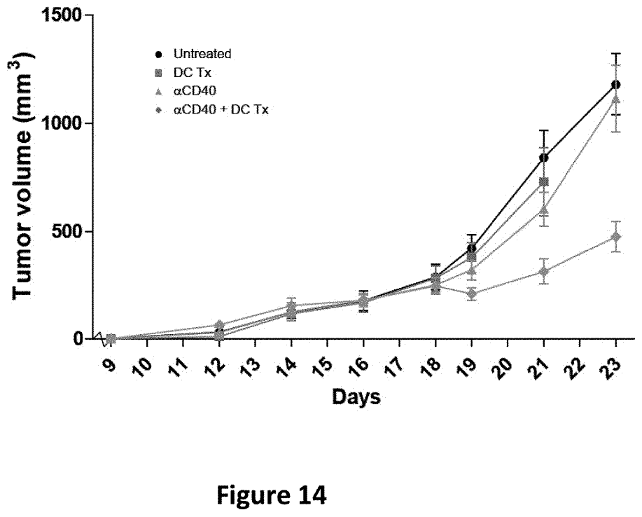

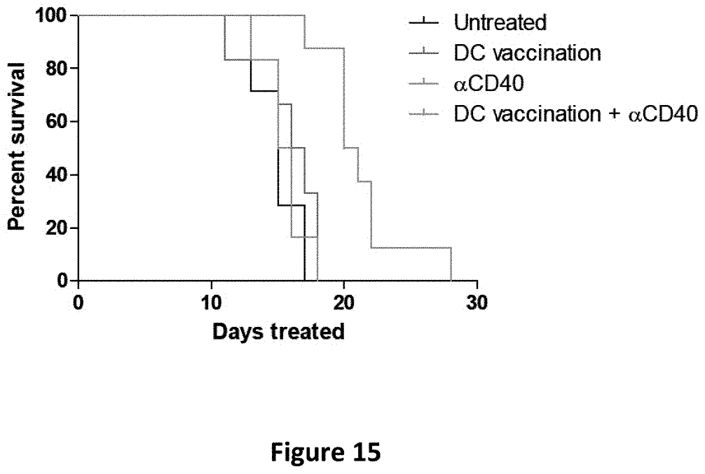

In order to determine if the positive effects observed with 5 day tumours would also be seen in larger tumours (10 days) a more intensified treatment schedule was used (FIG. 13). In this experimental setup, tumour growth and survival of mice treated with monotherapy DC vaccination (i.e. AE17) alone or CD40 agonistic monoclonal antibody alone (i.e. FGK45) did not significantly differ from untreated tumour-bearing mice. The combination therapy, however, significantly delayed tumour growth as shown in FIG. 14. It also resulted in an improved survival as shown in FIG. 15. Interim peripheral blood analysis demonstrated that both monotherapy DC vaccination and CD40 agonistic monoclonal antibody treatment induced higher frequencies of CD69+, Ki-67+ and PD-1+ T cells. Combination therapy induced higher frequencies of CD69+, Ki-67+ and PD-1+ for both CD4+ and CD8+ T cells.

### CONCLUSION

Reduced growth of established tumours was observed in mice treated with DC therapy in combination with CD40 agonist. DC therapy induced unique properties of immune cells in the circulation as well as in the tumour. This was mainly present in the CD4+ T cell compartment. The addition of CD40 agonistic monoclonal antibody potentiates DC vaccination leading to a significantly reduced tumour growth compared to untreated mice. This was not seen in mice treated with agonistic CD40 monotherapy or DC treatment alone. This may be a result of modulation of the characteristic desmoplastic stroma in pancreatic cancer leading to the influx of tumour-specific T cells.

### REFERENCES

1. Fest J, Ruiter R, van Rooij F J, van der Geest L G, Lemmens V E, lkram M A, et al. Underestimation of pancreatic cancer in the national cancer registry - Reconsidering the incidence and survival rates. Eur J Cancer. 2017; 72:186-91.
2. Rahib L, Smith B D, Aizenberg R, Rosenzweig A B, Fleshman J M, Matrisian L M. Projecting cancer incidence and deaths to 2030: the unexpected burden of thyroid, liver, and pancreas cancers in the United States. Cancer Res. 2014; 74(11):2913-21.
3. (IKNL), N.C.C.N., Pancreas—en periampullair carcinoom.—Kankerzorg in beeld. 2014.
4. Cleary S P, Gryfe R, Guindi M, Greig P, Smith L, Mackenzie R, et al. Prognostic factors in resected pancreatic adenocarcinoma: analysis of actual 5-year survivors. J Am Coll Surg. 2004; 198(5):722-31.
5. Paniccia A, Hosokawa P, Henderson W, Schulick R D, Edil B H, McCarter M D, et al. Characteristics of 10-Year Survivors of Pancreatic Ductal Adenocarcinoma. JAMA Surg. 2015; 150(8):701-10.
6. Neoptolemos J P, Palmer D H, Ghaneh P, Psarelli E E, Valle J W, Halloran C M, et al. Comparison of adjuvant gemcitabine and capecitabine with gemcitabine monotherapy in patients with resected pancreatic cancer (ESPAC-4): a multicentre, open-label, randomised, phase 3 trial. Lancet (London, England). 2017; 389(10073):1011-24.
7. Loehrer P P M, Cardenes H R, et al. A randomized phase III study of gemcitabine in combination with radiation therapy versus gemcitabine alone in patients with localized, unresectable pancreatic cancer. E4201 J Clin Oncol Suppl(abstract 4506). 2008.
8. Chen J, Guo X Z, Li H Y, Liu X, Ren L N, Wang D, et al. Generation of CTL responses against pancreatic cancer in vitro using dendritic cells co-transfected with MUC4 and survivin RNA. Vaccine. 2013; 31(41):4585-90.
9. Lepisto A J, Moser A J, Zeh H, Lee K, Bartlett D, McKolanis J R, et al. A phase I/II study of a MUC1 peptide pulsed autologous dendritic cell vaccine as adjuvant therapy in patients with resected pancreatic and biliary tumors. Cancer Ther. 2008; 6(B):955-64.
10. Poch B, Lotspeich E, Ramadani M, Gansauge S, Beger H G, Gansauge F. Systemic immune dysfunction in pancreatic cancer patients. Langenbecks Arch Surg. 2007; 392(3):353-8.
11. Ikemoto T, Yamaguchi T, Morine Y, Imura S, Soejima Y, Fujii M, et al. Clinical roles of increased populations of Foxp3+CD4+ T cells in peripheral blood from advanced pancreatic cancer patients. Pancreas. 2006; 33(4):386-90.
12. Carstens J L, Correa de Sampaio P, Yang D, Barua S, Wang H, Rao A, et al. Spatial computation of intratumoral T cells correlates with survival of patients with pancreatic cancer. Nat Commun. 2017; 8:15095.
13. Aerts J G, Hegmans J P. Tumor-specific cytotoxic T cells are crucial for efficacy of immunomodulatory antibodies in patients with lung cancer. Cancer Res. 2013; 73(8):2381-8.
14. Royal R E, Levy C, Turner K, Mathur A, Hughes M, Kammula U S, et al. Phase 2 trial of single agent Ipilimumab (anti-CTLA-4) for locally advanced or metastatic pancreatic adenocarcinoma. J Immunother. 2010; 33(8):828-33.
15. Aglietta M, Barone C, Sawyer M B, Moore M J, Miller W H, Jr., Bagala C, et al. A phase I dose escalation trial of tremelimumab (CP-675,206) in combination with gemcitabine in chemotherapy-naive patients with metastatic pancreatic cancer. Ann Oncol. 2014; 25(9):1750-5.
16. Brahmer J R, Tykodi S S, Chow L Q, Hwu W J, Topalian S L, Hwu P, et al. Safety and activity of anti-PD-L1 antibody in patients with advanced cancer. N Engl J Med. 2012; 366(26):2455-65.
17. Papaioannou N E, Beniata O V, Vitsos P, Tsitsilonis O, Samara P. Harnessing the immune system to improve cancer therapy. Ann Transl Med. 2016; 4(14):261.
18. Dammeijer F, Lievense L A, Veerman G D, Hoogsteden H C, Hegmans J P, Arends L R, et al. Efficacy of Tumor Vaccines and Cellular Immunotherapies in Non-Small- Cell Lung Cancer: A Systematic Review and Meta-Analysis. J Clin Oncol. 2016; 34(26):3204- [symbol]
19. Oyasiji T, Ma V W V. Novel adjuvant therapies for pancreatic adenocarcinoma. Journal of Gastrointestinal Oncology. 2015; 6(4):430-5.
20. D. C. NewLink Genetics Announces Results from Phase 3 IMPRESS Trial of Algenpantucel-L for Patients with Resected Pancreatic Cancer. Press Release: Globe Newswire: NewLink Genetics Corporation. 2016.
21. Lutz E, Yeo C J, Lillemoe K D, Biedrzycki B, Kobrin B, Herman J, et al. A lethally irradiated allogeneic granulocyte-macrophage colony stimulating factor-secreting tumor vaccine for pancreatic adenocarcinoma. A Phase II trial of safety, efficacy, and immune activation. Ann Surg. 2011; 253(2):328-35.
22. Le D T, Wang-Gillam A, Picozzi V, Greten T F, Crocenzi T, Springett G, et al. Safety and survival with GVAX pancreas prime and Listeria Monocytogenes-expressing mesothelin (CRS-207) boost vaccines for metastatic pancreatic cancer. J Clin Oncol. 2015; 33(12):1325-33.
23. Jaffee E M, Hruban R H, Biedrzycki B, Laheru D, Schepers K, Sauter P R, et al. Novel allogeneic granulocyte-macrophage colony-stimulating factor-secreting tumor vaccine for pancreatic cancer: a phase I trial of safety and immune activation. J Clin Oncol. 2001; 19(1):145-56.
24. Dung L, et al (2017). Results from a phase 2b, randomized, multicenter study of GVAX pancreas and CRS-207 compared to chemotherapy in adults with previously-treated metastatic pancreatic adenocarcinoma (ECLIPSE Study). Journal of Clinical Oncology. 35. 345-345.
25. Rosenberg S A, Restifo N P. Adoptive cell transfer as personalized immunotherapy for human cancer. Science. 2015; 348(6230):62-8.
26. Ryschich E, Notzel T, Hinz U, Autschbach F, Ferguson J, Simon I, et al. Control of T-cell-mediated immune response by HLA class I in human pancreatic carcinoma. Clin Cancer Res. 2005; 11(2 Pt 1):498-504.
27. von Bernstorff W, Voss M, Freichel S, Schmid A, Vogel I, Johnk C, et al. Systemic and local immunosuppression in pancreatic cancer patients. Clin Cancer Res. 2001; 7(3 Suppl):925s-32s.
28. Klebanoff C A, Rosenberg S A, Restifo N P. Prospects for gene-engineered T cell immunotherapy for solid cancers. Nat Med. 2016; 22(1):26-36. [symbol] immunotherapeutics. Immunotherapy. 2011; 3(4):517-37.
29. Johnson L A, Morgan R A, Dudley M E, Cassard L, Yang J C, Hughes M S, et al. Gene therapy with human and mouse T-cell receptors mediates cancer regression and targets normal tissues expressing cognate antigen. Blood. 2009; 114(3):535-46.
30. Lamers C H, Sleijfer S, van Steenbergen S, van Elzakker P, van Krimpen B, Groot C, et al. Treatment of metastatic renal cell carcinoma with CAIX CAR-engineered T cells: clinical evaluation and management of on-target toxicity. Mol Ther. 2013; 21(4):904-12.
31. Parkhurst M R, Yang J C, Langan R C, Dudley M E, Nathan D A, Feldman S A, et al. T cells targeting carcinoembryonic antigen can mediate regression of metastatic colorectal cancer but induce severe transient colitis. Mol Ther. 2011; 19(3):620-6.
32. Beatty G L, Haas A R, Maus M V, Torigian D A, Soulen M C, Plesa G, et al. Mesothelin-specific chimeric antigen receptor mRNA-engineered T cells induce anti-tumor activity in solid malignancies. Cancer Immunol Res. 2014; 2(2):112-20.
33. Linette G P, Stadtmauer E A, Maus M V, Rapoport A P, Levine B L, Emery L, et al. Cardiovascular toxicity and titin cross-reactivity of affinity-enhanced T cells in myeloma and melanoma. Blood. 2013; 122(6):863-71.
34. Morgan R A, Chinnasamy N, Abate-Daga D, Gros A, Robbins P F, Zheng Z, et al. Cancer regression and neurological toxicity following anti-MAGE-A3 TCR gene therapy. J Immunother. 2013; 36(2):133-51.
35. Debets R, Donnadieu E, Chouaib S, Coukos G. TCR-engineered T cells to treat tumors: Seeing but not touching? Semin Immunol. 2016; 28(1):10-21.
36. Banchereau J, Steinman R M. Dendritic cells and the control of immunity. Nature. 1998; 392(6673):245-52.
37. Hansen S G, Wu H L, Burwitz B J, Hughes C M, Hammond K B, Ventura A B, et al. Broadly targeted CD8(+) T cell responses restricted by major histocompatibility complex E. Science. 2016; 351(6274):714-20.
38. Heuvers M E, Aerts J G, Cornelissen R, Groen H, Hoogsteden H C, Hegmans J P. Patient-tailored modulation of the immune system may revolutionize future lung cancer treatment. BMC Cancer. 2012; 12:580.
39. Cornelissen R, Lievense L A, Heuvers M E, Maat A P, Hendriks R W, Hoogsteden H C, et al. Dendritic cell-based immunotherapy in mesothelioma. Immunotherapy. 2012; 4(10):1011-22.
40. DeMatos P, Abdel-Wahab Z, Vervaert C, Seigler HF. Vaccination with dendritic cells inhibits the growth of hepatic metastases in B6 mice. Cell Immunol. 1998; 185(1):65-74.
41. Fields R C, Shimizu K, Mule J J. Murine dendritic cells pulsed with whole tumor lysates mediate potent antitumor immune responses in vitro and in vivo. Proc Natl Acad Sci U S A. 1998; 95(16):9482-7.
42. Palucka A K, Ueno H, Connolly J, Kerneis-Norvell F, Blanck J P, Johnston D A, et al. Dendritic cells loaded with killed allogeneic melanoma cells can induce objective clinical responses and MART-1 specific CD8+ T-cell immunity. J Immunother. 2006; 29(5):545-57.
43. Thomas-Kaskel A K, Zeiser R, Jochim R, Robbel C, Schultze-Seemann W, Waller C F, et al. Vaccination of advanced prostate cancer patients with PSCA and PSA peptide-loaded dendritic cells induces DTH responses that correlate with superior overall survival. Int J Cancer. 2006; 119(10):2428-34.
44. Pecher G, Haring A, Kaiser L, Thiel E. Mucin gene (MUC1) transfected dendritic cells as vaccine: results of a phase I/II clinical trial. Cancer Immunol Immunother. 2002; 51(11-12):669-73.
45. Stift A, Friedl J, Dubsky P, Bachleitner-Hofmann T, Schueller G, Zontsich T, et al. Dendritic cell-based vaccination in solid cancer. J Clin Oncol. 2003; 21(1):135-42.
46. Kondo H, Hazama S, Kawaoka T, Yoshino S, Yoshida S, Tokuno K, et al. Adoptive immunotherapy for pancreatic cancer using MUC1 peptide-pulsed dendritic cells and activated T lymphocytes. Anticancer Res. 2008; 28(1B):379-87.
47. Koido S, Homma S, Okamoto M, Takakura K, Mori M, Yoshizaki S, et al. Treatment with chemotherapy and dendritic cells pulsed with multiple Wilms' tumor 1 (WT1)—specific MHC class I/II-restricted epitopes for pancreatic cancer. Clin Cancer Res. 2014; 20(16):4228-39.
48. Mayanagi S, Kitago M, Sakurai T, Matsuda T, Fujita T, Higuchi H, et al. Phase I pilot study of Wilms tumor gene 1 peptide-pulsed dendritic cell vaccination combined with gemcitabine in pancreatic cancer. Cancer Sci. 2015; 106(4):397-406.
49. Mehrotra S, Britten C D, Chin S, Garrett-Mayer E, Cloud C A, Li M, et al. Vaccination with poly(IC:LC) and peptide-pulsed autologous dendritic cells in patients with pancreatic cancer. J Hematol Oncol. 2017; 10(1):82.
50. Bauer C, Dauer M, Saraj S, Schnurr M, Bauernfeind F, Sterzik A, et al. Dendritic cell-based vaccination of patients with advanced pancreatic carcinoma: results of a pilot study. Cancer Immunol Immunother. 2011; 60(8):1097-107.
51. Suso E M, Dueland S, Rasmussen A M, Vetrhus T, Aamdal S, Kvalheim G, et al. hTERT mRNA dendritic cell vaccination: complete response in a pancreatic cancer patient associated with response against several hTERT epitopes. Cancer Immunol Immunother. 2011; 60(6):809-18.
52. Amedei A, Niccolai E, Prisco D. Pancreatic cancer: role of the immune system in cancer progression and vaccine-based immunotherapy. Hum Vaccin Immunother. 2014; 10(11):3354-68.
53. Okamoto M, Kobayashi M, Yonemitsu Y, Koido S, Homma S. Dendritic cellbased vaccine for pancreatic cancer in Japan. World J Gastrointest Pharmacol Ther. 2016; 7(1):133-8.
54. Aerts J G J V, de Goeje P L, Cornelissen R, Kaijen-Lambers M E H, Bezemer K, van der Leest C H, Mahaweni N M, Kunert A, Eskens F A L M, Waasdorp C, Braakman E, van der Holt B, Vulto A G, Hendriks R W, Hegmans J P J J, Hoogsteden H C. Autologous Dendritic Cells Pulsed with Allogeneic Tumor Cell Lysate in Mesothelioma: From Mouse to Human. Clin Cancer Res. 2018 Feb. 15; 24(4):766-776
55. Carreno B M, Magrini V, Becker-Hapak M, Kaabinejadian S, Hundal J, Petti A A, Ly A, Lie W R, Hildebrand W H, Mardis E R, Linette G P. Cancer immunotherapy. A dendritic cell vaccine increases the breadth and diversity of melanoma neoantigen-specific T cells. Science. 201; 348(6236):803-8
56. Ho M Y, Tang S J, Sun K H, Yang W. Immunotherapy for lung cancers. J Biomed Biotechnol. 2011; 2011:250860
57. Hegmans J P, Veltman J D, Lambers M E, de Vries I J, Figdor C G, Hendriks R W, Hoogsteden H C, Lambrecht B N, Aerts J G. Consolidative dendritic cell-based immunotherapy elicits cytotoxicity against malignant mesothelioma. Am J Respir Crit Care Med. 2010 Jun. 15; 181(12):1383-90.
58. Banchereau J, Palucka A K. Dendritic cells as therapeutic vaccines against cancer. Nat Rev Immunol. 2005 April; 5(4):296-306.
59. Berger T G, Strasser E, Smith R, Carste C, Schuler-Thurner B, Kaempgen E, Schuler G. Efficient elutriation of monocytes within a closed system (Elutra) for clinical-scale generation of dendritic cells. J Immunol Methods. 2005 March; 298(1-2):61-72.
60. Magnusson A, Laurell A, Lonnemark M, Brekkan E, Adamson L, Tolf A, Andersson B, Wallgren A, Kiessling R, and Karlsson-Parra A. Intratumoral vaccination with activated allogeneic dendritic cells in patients with newly diagnosed metastatic renal cell carcinoma (mRCC). J Clin Oncol. 2014; 32:15_suppl:3085-3085
61. Vonderheide R H, Burg J M, Mick R, et al. Phase I study of the CD40 agonist antibody CP-870,893 combined with carboplatin and paclitaxel in patients with advanced solid tumors. Oncoimmunology. 2013; 2(1):e23033
62. Vitale L A, Thomas L J, He L Z, O'Neill T, Widger J, Crocker A, Sundarapandiyan K, Storey J R, Forsberg E M, Weidlick J, Baronas A R, Gergel L E, Boyer J M, Sisson C, Goldstein J, Marsh H C Jr, Keler T. Development of CDX-1140, an agonist CD40 antibody for cancer immunotherapy. Cancer Immunol Immunother. 2018 Oct. 31. doi: 10.1007/s00262-018-2267-0. [Epub ahead of print]
63. Habib M, Noval Rivas M, Chamekh M, Wieckowski S, Sun W, Bianco A, Trouche N, Chaloin O, Dumortier H, Goldman M, Guichard G, Fournel S, Vray B. Cutting edge: small molecule CD40 ligand mimetics promote control of parasitemia and enhance T cells producing IFN-gamma during experimental Trypanosoma cruzi infection. J Immunol. 2007 Jun. 1; 178(11):6700-4.)
64. Morrison A H, Byrne K T, Vonderheide R H. Immunotherapy and Prevention of Pancreatic Cancer. Trends Cancer. 2018 June; 4(6):418-428.
65. Vonderheide R H, Bajor D L, Winograd R, Evans R A, Bayne L J, Beatty GL. CD40 immunotherapy for pancreatic cancer. Cancer Immunol Immunother. 2013 May; 62(5):949-54.

1. A method for the treatment of pancreatic cancer, comprising administering to a patient in need thereof a CD40 agonist in combination with dendritic cells loaded with a lysate, wherein the lysate is obtainable by a method comprising:
(i) providing human mesothelioma tumour cells from at least two different mesothelioma tumour cell lines;
(ii) inducing necrosis in the tumour cells; and
(iii) lysing the necrotic tumour cells, such that a lysate is obtained.

2. The method according to claim 1, wherein the pancreatic cancer is unresected pancreatic cancer.

3. The method according to claim 1, wherein the pancreatic cancer is primary pancreatic cancer, locally advanced pancreatic cancer, metastatic pancreatic cancer or borderline resectable pancreatic cancer.

4. The method according to claim 1, wherein the pancreatic cancer is recurrent pancreatic cancer.

5. The method according to claim 1, wherein the CD40 agonist is administered to the patient after the dendritic cells have been administered.

6. The method according to claim 1, wherein the CD40 agonist is chosen from the group consisting of CP-870, CP-893, CDX-1140, APX005M, RG7876/selicrelumab, ADC-1013/JNJ-64457107, ABBV-428, SEA-CD40 and MED15083.

7. The method according to claim 1, wherein induction of necrosis of the mesothelioma tumour cells is achieved by subjecting the cells to freeze-thawing cycles.

8. The method according to claim 1, further comprising (iv) subjecting the lysate obtained to at least 50 Gy.

9. The method according to claim 1, further comprising (iv) subjecting the lysate obtained to at least 100 Gy.

10. The method according to claim 1, wherein the mesothelioma tumour cells provided comprise tumour cells from at least three mesothelioma tumour cell lines.

11. The method according to claim 1, wherein the mesothelioma tumour cells are chosen from two or more of the following cell lines: Thorr 01 (deposit No. DSM ACC3192), Thorr 02 (deposit No. DSM ACC3193), Thorr 03 (deposit No. DSM ACC3191), Thorr 05 (deposit No. DSM ACC3194), Thorr 06 (deposit No. DSM ACC3195).

12. The method according to claim 1, wherein the lysate comprises at least three mesothelioma cancer cell associated antigens.

13. The method according to claim 11, wherein the mesothelioma cancer cell associated antigens are selected from the group consisting of RAGE1/MOK, Mesothelin, EphA2, Survivin, WT1, MUC1, RAB38/NY-MEL-1, BING4, MAGE A12, HER-2/Neu, Glypican, and LMP2.

14. The method according to claim 1, wherein the lysate is loaded onto autologous dendritic cells of the patient.

15. The method according to claim 1, wherein the dendritic cells are loaded with between 1 tumour cell equivalents per 100 dendritic cells to 10 tumour cell equivalents per 1 dendritic cell.

16. The method according to claim 1, wherein 1*106 to 1*109 loaded dendritic cells are administered per dose.

17. A pharmaceutical composition, obtainable by a method comprising:
(i) preparing a lysate of allogeneic mesothelioma tumour cells from at least two different cell lines; and
(ii) loading dendritic cells with the lysate of tumour cells and, optionally, adding a pharmaceutically acceptable carrier.

18. The pharmaceutical composition according to claim 17, comprising 1*106 to 1*109 dendritic cells.

19. The pharmaceutical composition according to claim 17, wherein the dendritic cells are loaded with between 1 tumour cell equivalents per 100 dendritic cells to 10 tumour cell equivalents per 1 dendritic cell.

20. The pharmaceutical composition according to claim 17, wherein the composition further comprises an adjuvant.
